(19) United States
(12) Patent Application Publication
(10) Pub. No.: US 2005/0137164 A1
(43) Pub. Date: Jun. 23, 2005

(54) Diclofenac compositions for the treatment of skin disorders

(76) Inventors: Moshe Arkin, Kfar Shmaryahu (IL); Amira Zeevi, Omer (IL); Stephen Cherkez, Caesarea (IL); Eilon Asculai, Lehavim (IL); Chalil Abu-Gnim, Beer Sheva (IL); Ido Yosha, M.P. Hanegev (IL); Michal Arnon, Sde Boker (IL); Hila Ohayon-Tsahor, Sde Boker (IL); Oren Chen, Beer Sheva (IL); Galia Fridler, Modiin (IL)

Correspondence Address: Martin Moynihan;c/o ANTHONY CASTORINA, SUITE 207, 2001 JEFFERSON DAVIS HIGHWAY, ARLINGTON, VA, 22202, US

(21) Appl. No.: 10/946,560

(22) Filed: Sep. 22, 2004

Related U.S. Application Data
(60) Provisional application No. 60/503,883, filed on Sep. 22, 2003.

(51) Int. Cl. A61K 31/195

(52) U.S. Cl. 514/54; 514/567; 424/448

(57) ABSTRACT

Novel NSAID pharmaceutical compositions and methods for the treatment of skin disease and disorders such as actinic keratosis using same are disclosed.

This application claims the benefit of priority from U.S. Provisional Patent Application No. 60/503,883, filed Sep. 22, 2003.

### FIELD AND BACKGROUND OF THE INVENTION

The present invention relates to novel compositions for the treatment of skin diseases and disorders, and more particularly, to novel gel compositions of a non-steroidal anti-inflammatory drug such as diclofenac or a pharmaceutically acceptable salt thereof, and their use in such a treatment.

The skin is the largest organ of the body, covering the entire outside of the body, and comprises the epidermis, dermis, and subcutaneous layers. Numerous disorders of the skin are known, ranging form those which merely cause discomfort or psychological stress, such as rashes, to those which are life threatening, such as skin cancer.

Skin cancer is the most common form of cancer in the United States. Skin cancers are classified by the types of epidermal cells involved. Basal cell carcinoma develops from abnormal growth of the cells in the lowest layer of the epidermis and is the most common type of skin cancer; squamous cell cancer involves changes in the squamous cells, found in the middle layer of the epidermis; and melanoma occurs in the melanocytes. Melanoma is less common than squamous or basal cell carcinoma, but more dangerous. It is the leading cause of death from skin disease.

Actinic keratosis is a precancerous skin growth usually caused by sun exposure, which may develop into squamous cell cancer. Actinic keratosis lesions are the most common neoplastic skin lesions detected in individuals with Fitzpatrick skin type I or II. Actinic keratosis lesions appear as papules in a vast spectrum of sizes, shapes, colors, and other characteristics. Their size and shape can range from a well-circumscribed, single millimeter papule to an irregularly shaped lesion that can span several centimeters. These neoplasms can be flesh color, red or pigmented and also can scale or become hyperkeratotic. The most common sites for these lesions are the face, ears, scalp, neck, forearms, and hands.

To combat this very common skin disorder, a host of topical preparations has been investigated. One of the presently existing therapies includes diclofenac preparations, whereby others include, for example, fluorouracil, imiquimod, colchicine and retinoids (Tutrone et al., Cont. Med. Edu. 71: 373-379, 2003).

Diclofenac is a non-steroidal anti-inflammatory drug. Non-steroidal anti-inflammatory drugs (NSAIDs) are drugs having analgesic, antipyretic and anti-inflammatory effects, resulting in reduction of pain, fever and inflammation. They act by inhibiting cyclooxygenase enzymes, thereby reducing the conversion of arachidonic acid to prostaglandins. Most known NSAIDs act as non-selective inhibitors of cyclooxygenase, i.e. they inhibit both the cyclooxygenase-1 (COX-1) and cyclooxygenase-2 (COX-2) isoenzymes.

In a search for drug treatment of skin diseases or disorders, the role of skin in drug delivery is considered. The skin can be used for drug delivery in two ways, transdermally and dermally. While transdermal drug delivery typically involves percutaneous delivery of the drug across the skin into the systemic circulation (the blood steam), drugs are ideally formulated and administered in such a way as to enable an optimal concentration of active agent to be delivered to the intended target site. This is typically achieved by incorporating one or more compounds that act as penetration enhancers in a formulation for transdermal delivery of a drug. Penetration enhancers are materials that have a direct effect on the permeability of one or more of the skin layers. Chemical penetration enhancers are believed to operate mainly in the intercellular spaces of the stratum corneum. The exact mechanisms by which many chemical penetration enhancers function have not been clearly elucidated; it is almost certain that they will have multiple effects once absorbed into the stratum corneum. Effects a have been documented include an alteration of the solvent potential of the stratum corneum's biochemical environment, and a disordering of the intercellular lipid matrix following insertion of the enhancer into the bilayer structure.

In dermal drug delivery, however, the skin itself is the target organ and hence an ideal delivery profile for a therapeutically active agent for treating skin disorders such as actinic keratosis should involve localization of the topically applied drug in the skin, with little or no systemic absorption of the drug. Such a desired localization may be achieved by inclusion in a formulation containing the therapeutically active agent, of an agent that is capable of modifying the transdermal penetration of the therapeutically active agent. Such an agent is oftentimes referred to in the art as a penetration modifier.

In contrast to penetration enhancers, which increase the transdermal delivery of an active agent, penetration modifiers have the opposite effect, such that transport of the active agent across the skin barrier and into the system is minimized, thereby increasing the time during which the agent is in contact with the affected layer of the skin. Penetration modifiers typically alter the distribution and performance of the administered drug in the skin and/or exposed tissue, particularly the epidermis, and produce an unusual targeting for underperfused skin and/or pathological tissue in the skin (site of trauma and/or pathology). Thus, in the presence of penetration modifiers, the administered drug passes into the skin, accumulates and stays longer in the skin at the site of the trauma and/or pathology.

One of the presently known agents that may provide such a desired effect in dermal drug delivery is hyaluronic acid. Hyaluronic acid was found to be advantageous in the dermal delivery and localization of drugs in the skin, by causing modified transdermal penetration of the drugs, thereby allowing for a prolonged stay of the drug at the treatment site (Brown, International Journal of Pharmaceutics, 225: 113-121,2001).

Thus, U.S. Pat. Nos. 5,639,738, 5,985,950, 5,929,048, 5,792,753, 5,852,002, 5,914,322 and 6,147,059, to Falk et al., teach the use of topical mixtures containing up to 3% by weight NSAIDs, in compositions essentially comprised of hyaluronic acid or a pharmaceutically acceptable salt thereof, in a molecular weight range of between 150,000 to 750,000 Daltons, for the treatment of various skin disorders, including actinic keratosis. In these patents it is taught that although higher molecular weights of the hyaluronic acid and forms thereof may be used to penetrate the skin and/or exposed tissue, and transport the medicines or drugs, fragments of lesser molecular weight are preferred. Hence, according to the teachings of these patents, in cases where the molecular weight of the hyaluronic acid chosen for use is very large, it is preferred that the form of hyaluronic acid is autoclaved, to thereby break down the hyaluronic acid to fragments of lesser molecular weight, or, if feasible, diluted to permit admiration and ensure no coagulation on or in the skin. In cases where the molecular weight of the form of hyaluronic acid being employed is large, the concentration of the form of the hyaluronic acid in the composition may, for example, be reduced (for example to less than about 3%) dependent on its molecular weight.

The topical mixtures disclosed in the patents to Falk et al. are intended to be applied several times daily, over a period of 3-4 weeks, directly upon the affected skin areas, to help break down and clear lesions.

The patents to Falk et al. further emphasize the need for hyaluronic acid, particularly in the molecular weight range of between 150,000 to 750,000 Daltons, for obtaining and facilitating the desired therapeutic effect. Hyaluronic acid and salts thereof, are taught as altering the distribution and performance of drugs such as NSAIDs in the skin and/or exposed tissue, particularly the epidermis, and producing an unusual targeting for underperfused skin and/or pathological tissue in the skin.

Falk et al. further teach that the effective non-toxic dosage amount of the form of hyaluronic acid and the effective dosage amount of NSAID passing through the stratum corneum to the epidermis and to the dermis, passes into the skin, accumulating and staying longer in the skin at the site of the trauma and/or pathology. Therefore, after having had an immediate effect at the site of trauma and/or pathology (for example, relieving pain and acting on the basal cell carcinoma, actinic keratosis and other disease, condition or lesion), the NSAID-hyaluronic acid combination continues to accumulate at the site in need of treatment and thereafter clears through the lymphatic system.

These recitations imply that formulations devoid of hyaluronic acid would be less therapeutically effective (if at all), and as a result would require higher concentrations of NSAID, or would exhibit undesired systemic effects.

Solaraze™ is the trade name of a commercially available product, approved by the FDA for topical use in treating actinic keratosis, currently marketed by Bioglan Pharma, Inc. Solaraze™ is a gel formulation containing 3% by weight of diclofenac sodium as an active ingredient and 2.5% hyaluronate sodium, and is directly applied twice daily to the immediate areas of the actinic keratosis. The hyaluronic acid salt in this product has a molecular weight that ranges between 150,000 and 750,000 Daltons.

Nevertheless, although hyaluronic acid was found useful when incorporated into formulations containing diclofenac for treating actinic keratosis, hyaluronic acid is known as a highly expensive reagent, having exact specifications and a limited number of suppliers. Hence, the inclusion of hyaluronic acid in such formulations is highly limited by its high cost, low commercial availability and exacting specifications.

In view of these limitations, it is widely recognized that compositions for treating skin diseases and disorders, particularly compositions containing NSAIDs such as diclofenac as the active ingredient, which are essentially free of hyaluronic acid, are highly desirable.

NSAID compositions, essentially free of hyaluronic acid, have been described in the art.

U.S. Patent Application Publication No. 20030004143, to Prior et al., for example, teaches NSAID compositions which are useful for the treatment and prevention of cellular abnormalities of organs of the female reproductive tract and particularly of the cervix, vagina and vulva. Inherently, the indications targeted include, e.g., cervix cancer and the like and do not include skin disorders such as actinic keratosis. Moreover, the reproductive tract, unlike the skin, is a mucosal membrane which is easily penetrated, especially in comparison with the stratum corneum. While this patent application recites diclofenac as one of a long list of NSAIDs that act as COX-1 inhibitors, preferred formulations include keratolac as the NSAID. This patent application therefore fails to teach NSAID compositions, and particularly diclofenac compositions, that can be beneficially used in the treatment of skin diseases or disorders such as actinic keratosis.

Topical combinations of alpha-difluoromethylornithine (alpha-DFMO) and NSAIDs, including diclofenac, for the treatment of actinic keratosis are described in U.S. Patent Application Publication No. 20030129208 to Alberts et al. According to the teachings of this patent application, alpha-DFMO is combined with an NSAID such as diclofenac so as to decrease intracellular levels of putrescine and spermidine in the skin. Alpha-DFMO is taught as being an essential active ingredient in the formulations taught by this patent application, whereby addition of a steroidal and/or a non-steroidal anti-inflamatory drug NSAID to these formulations results in a synergistic effect. This patent application therefore fails to teach such formulations, in which an NSAID acts as the active ingredient alone. Moreover, since alpha-DFMO is known as a neoplastic agent, its inclusion as an essential active ingredient is oftentimes associated with adverse side effects and hence formulations that are devoid tehrcof would be highly advantageous.

U.S. Patent Application Publication No. 20030143165 to Evans et al. discloses NSAID-containing formulations for the prevention of non-melanoma skin cancer, for use over a long period of time, measured in years. These formulations are intended to prevent development of skin cancers in, e.g., patients exposed to ultraviolet light, and are therefore not intended for use in the treatment of skin cancers. As such, the taught formulations include relatively low concentration of an NSAID of up to 2 weight percent, and are directed to be applied daily, on a regular basis, over large, non-affected areas of the skin.

Compositions containing NSAIDs such as diclofenac, together with components such as, urea, glycol monomethyl ether and glycerine, which have the potential to act as penetration enhancers, are also known in the art. However, the inclusion in the compositions of these compounds specifically for the purpose of penetration modulation is neither taught nor suggested.

Urea is a well-known ingredient of topical formulations. U.S. Pat. No. 5,874,479, to Martin, discloses the use of urea as a penetration enhancer for topical diclofenac formulations. U.S. Patent Application Publication No. 20020012695, to Wan et al., discloses urea as a penetration enhancer for transdermal patch formulations of diclofenac salts. Both those patents therefore refer to urea as a penetration enhancer, i.e. an ingredient that promotes and enhances the rate of transdermal drug absorption, allowing it to rapidly enter the systemic circulation. Takahashi et al., (Biol. Pharm. Bull., 1995) also teach that urea can be a penetration enhancing agent for diclofenac sodium transdermal compositions. Takahashi et al. teach that in cream or emulsion compositions, a concentration of 3% of urea enhanced the permeation of diclofenac sodium through the skin to the receptor medium.

Diethylene glycol monomethyl ether (Transcutol™) is another well-known ingredient of topical formulations. European Patent No. 804160, to Yissum, discloses the use of Transcutol™ as part of a liposomal composition for enhanced transdermal delivery of drugs, including NSAIDs. Other ingredients in the disclosed composition are phospholipids, lower alcohol and water. A topical delivery system using Transcutol™ was found useful for the dermal delivery of corticosteroids such as hydrocortisone and dexamethasone (Panchagnula, J. et al. Pharm. Pharmacol., 1991). Panchagnula et al. state that the optimal Transcutol™ concentration for the dermal delivery of the corticosteroids is 50%. Another study (Godwin et al. Euro. J. Pharm. Biopharm., 2002) showed that high concentrations of Transcutol™, of higher than 15% by weight, were necessary to achieve significant skin accumulation of UV absorbers oxybenzone and connamate. For optimal results, a concentration of 50% by weight of Transcutol™ was needed.

As it is well known and established that topical compositions containing Transcutol™ in concentrations greater than 10 weight percentages are highly undesirable (for example, the maximum topical concentration of Transcutol™ permitted by the FDA is 10% by weight), the use of such high concentrations of Transcutol™, as taught by these prior art references, is highly disadvantageous.

Polysorbate 80 (“Tween 80”™) was found to decrease the skin permeation of diclofenac sodium in gel compositions (Arellano et al. Eur J Drug Metab Pharmacokinet, 1998). However, although Arellano et al. discuss Polysorbate 80 regarding the transdermal delivery of diclofenac sodium, its effect on the dermal delivery of diclofenac or a pharmaceutically acceptable salt thereof was not investigated.

Glycerine is another well-known ingredient of topical formulations. European Patent No. 644746 to Heiber et al. discloses the use of glycerine as a moderator for the transdermal delivery of drugs. Heiber et al. teach that the use of a high concentration of glycerine moderates and maintains drug transdermal penetration over time, i.e. the delivery by passage of drug through the skin or mucosal tissue to the systemic circulation. The system disclosed in this patent is intended for use with transdermally delivered drugs, to delay the initial burst effect. Use of the system for dermal delivery is neither taught nor suggested by this prior art patent.

Hence, while the prior art teaches various NSAID-containing topical formulations, it fails to teach such compositions for the treatment of skin disorders, particularly actinic keratosis, which are free of hyaluronic acid and are therefore devoid of the limitations associated therewith.

### SUMMARY OF THE INVENTION

The present inventors have now surprisingly found that topical compositions that are essentially free of hyaluronic acid, and are therefore devoid of the limitations associated therewith are particularly effective systems for dermal delivery of NSAIDs such as diclofenac for treatment of skin disease or disorders in which dermal application of NSAID is beneficial.

Hence, according to one aspect of the present invention there is provided a pharmaceutical composition identified for use in the treatment of skin disease or disorders, which comprises non-steroidal anti-inflammatory drug (NSAID) or a pharmaceutically acceptable salt thereof and a pharmaceutically acceptable carrier, and which is essentially free of hyaluronic acid, and more specifically, essentially fee of hyaluronic acid having a molecular weight of between 150,000 to 750,000 Daltons.

The NSAIDs can be, for example, diclofenac, oxicams, piroxicam, meloxicam, isoxicam, tenoxicam, sudoxicam, CP-14,304, salicylates, aspirin, disalcid, benorylate, trilisate, safapryn, solprin, diflunisal fendosal, acetic acid derivatives, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof, and combinations thereof. More preferably, the non-steroidal anti-inflammatory drug is diclofenac or a pharmaceutically acceptable salt thereof, more preferably diclofenac sodium.

The presently most preferred NSAID according to the present invention is diclofenac and hence, according to another aspect of the present invention there is provided a pharmaceutical composition which comprises, as an active ingredient, diclofenac or a pharmaceutically acceptable salt thereof and a pharmaceutically acceptable carrier, and which is essentially free of hyaluronic acid, and more specifically, essentially free of hyaluronic acid having a molecular weight of between 150,000 to 750,000 Daltons.

As is demonstrated in the Examples section that follows, it was found that the compositions described above are highly active in treating skin disorders when diclofenac is incorporated therein as the sole active ingredient. Hence, according to still another aspect of the present invention, there are provided pharmaceutical compositions as described hereinabove, in which the NSAID (e.g., diclofenac) or its salt is the sole active ingredient of the pharmaceutical composition and/or which are devoid of alpha-difluoromethylornithine.

Skin diseases and disorders which are treatable by the compositions of the present invention include, for example, basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, or hair loss in pregnancy, preferably actinic keratosis.

According to further features in preferred embodiments of the invention described below, each of the pharmaceutical compositions of the present invention is devoid of a penetration modifier.

Alternatively, each of the pharmaceutical compositions of the present invention, described above, comprises at least one penetration modifier.

The penetration modifier is preferably selected from the group consisting of diethylene glycol monomethyl ether, urea, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof.

In cases where the pharmaceutical composition comprises diethylene glycol monoethyl ether as a penetration modifier, a concentration of the diethylene glycol monoethyl ether preferably ranges between about 5 weight percentages and about 15 weight percentages of the total weight of the composition, more preferably between about 5 weight percentages and about 10 weight percentages.

In cases where the pharmaceutical composition comprises urea as a penetration modifier, a concentration of the urea preferably ranges between about 5 weight percentages and about 15 weight percentages of the total weight of the composition, more preferably between about 5 weight percentages and about 10 weight percentages.

Pharmaceutical compositions according to the present invention which comprise urea may optionally and preferably further comprise a pH stabilizing agent selected from the group consisting of a hydroxyacid, allantoin, hydrochloric acid, a buffer system, an antioxidant and any mixture thereof.

In cases where the pharmaceutical composition comprises glycerine, a concentration of the glycerine preferably ranges between about 20 weight percentages and about 50 weight percentages of the total weight of the composition.

Such compositions may further comprise diethylene glycol monoethyl ether, whereby, preferably, the concentration of glycerine ranges between about 20 weight percentages and about 40 weight percentages, and the concentration of the diethylene glycol monoethyl ether ranges between about 5 weight percentages and about 15 weight percentages.

In cases where the pharmaceutical composition comprises Polysorbate 80 as a penetration modifier, a concentration of the Polysorbate 80 preferably ranges between about 1 weight percentage and about 5 weight percentages of the total weight of the composition, more preferably between about 2 weight percentages and about 3 weight percentages.

In cases where the pharmaceutical composition comprises a hyaluronic acid selected from the group consisting of hyaluronic acid or a salt thereof having a molecular weight of less than about 150,000 and hyaluronic acid or a salt thereof having a molecular weight greater than about 750,000, the concentration of the hyaluronic acid preferably ranges between about 1 weight percentage and about 3 weight percentages of the total weight of the composition, and more preferably is about 2.5 weight percentages.

According to still father features in the described preferred embodiments, the pharmaceutically acceptable salt is a sodium salt of the diclofenac or the NSAID.

The concentration of the diclofenac of the NSAID preferably ranges between about 1 weight percentage and about 5 weight percentages of the total weight of the composition, and more preferably is about 3 weight percentages.

Each of the pharmaceutical compositions described above can be in the form of a gel, a cream, an ointment, a paste, a lotion, a milk, a suspension, an aerosol, a spray, a foam, a serum, a swab, a pledglet, a pad or a patch, more preferably in the form of a gel.

Gel formulations according to the present invention preferably further comprise a gelling agent such as, for example, hydroxypropyl methylcellulose (HPMC) or hydroxyethylcellulose (HEC).

The concentration of the gelling agent preferably ranges between about 0.1 weight percentage and about 7 weight percentages of the total weight of the composition, more preferably between about 1 weight percentage and about 3 weight percentages.

Each of the pharmaceutical compositions of the present invention optionally further comprises an additive, such as, for example, a moisturizing agent, an emollient, a humectant, a deodorant agent, an antiperspirant, a pH adjusting agent, a preservative, an emulsifier, an occlusive agent, a solubilizing agent, a colorant, or a surfactant. Preferably, the concentration of the additive ranges between about 1 weight percentage and about 5 weight percentages of the total weight of the composition.

An exemplary preferred composition according to the present invention consists essentially of diclofenac sodium, preferably at a concentration of about 3 weight percentages; a gelling agent selected from the group consisting of hydroxypropyl methylcellulose and hydroxyethylcellulose; at least one penetration modifier selected from the group consisting of diethylene glycol monomethyl ether, ura, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof; at least one additive; and a pharmaceutically acceptable carrier.

The additive can be, for example, a preservative such as benzyl alcohol and/or a pH-stabilizing agent, in cases where the penetration modifier(s) comprise urea.

The carrier preferably comprises a polyalkylene glycol and water.

According to still further features in the described preferred embodiments each of the pharmaceutical compositions described above can be packaged in a packaging material and identified in print, in or on the package, for use in the treatment of the diseases or disorders described above.

Hence, according to an additional aspect of the present invention, there is provided a method for treating a skin disease or disorder, as described hereinabove, in a subject in need thereof, which comprises applying onto one or biological surfaces affected by the skin disease or disorder of the subject, a therapeutically effective amount of any of the pharmaceutical compositions described above.

Preferably, the biological surface is selected from the group consisting of the skin of the face, ears, scalp, neck, forearms, back, legs, arms and hands.

Further preferably, the composition is applied between 2 and 4 times a day, for a time period that ranges between about 20 days and about 120 days. More preferably, the composition is applied twice a day, for a time period that ranges between 30 and 60 days.

Further preferably, the subject is a human.

The compositions of the present invention were surprisingly found to have a similar or better therapeutic effect than that of an otherwise identical composition comprising hyaluronic acid of molecular weight in the range of from about 150,000 to about 750,000 Daltons.

The compositions of the present invention may further comprise commonly used ingredients such as urea, diethylene glycol monomethyl ether (Transcutol™), glycerine, polysorbate 80 (Tween™ 80), or combinations thereof, as penetration modifying agents. These materials were surprisingly found to have skin penetration modifying effects similar to those of hyaluronic acid, while being both more readily available and considerably cheaper. Hence, it was found that topical compositions, comprising an NSAID, and particularly diclofenac, or a salt thereof preferably as a sole active ingredient, in combination with these ingredients, which are essentially free of hyaluronic acid, have similar or better therapeutic performance than that of an NSAID composition comprising hyaluronic acid. Such compositions have obvious commercial and industrial advantages.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The phrase “applying” describes topical application onto one or more biological surface(s), by contacting a composition with the surface. Non-limiting examples of biological surfaces onto which the compositions of the present invention can be beneficially applied include one or more of the face, ears, scalp, neck, forearms, back, legs, arms and hands, depending on the skin condition that is being treated.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “pharmaceutically active ingredient” or “active ingredient” refers to a pharmaceutical agent including any natural or synthetic chemical substance that subsequent to its application has, at the very lest, at least one desired pharmaceutical effect.

“Carriers” or “vehicles” refer to carrier materials suitable for dermal drug administration and include any such material known in the art, e.g. any liquid, gel, solvent, liquid diluent, solubilizer or the like, which is nontoxic and which does not interact with other components of the composition in a deleterious manner. Examples of suitable carriers include water, alcohols, mineral oil, silicone, liquid sugars, waxes, petroleum jelly, and a variety of other oils and polymeric materials.

The term “therapeutically effective amount” or “pharmaceutically effective amount” denotes that dose of a pharmaceutically active ingredient or a composition comprising the pharmaceutically active ingredient that will provide the pharmacological effect for which the active ingredient is indicated.

As used herein, the phrase “Pharmaceutically acceptable carrier” describes a carrier that does not cause significant irritation to an organism and does not abrogate the biological activity and properties of the applied active ingredient.

As used herein, the phrase “penetration modifier”, which is also referred to herein interchangeably as “penetration modifying agent” describes one or more compounds that are capable of modifying the transdermal penetration of an active ingredient (a drug), preferably in such a way that the active ingredient is accumulated in the affected skin layer and thus the time during which the active ingredient is in contact with the affected skin layer is increased while the transdermal penetration of the active ingredient into the blood system is minimized. In other words, these terms are used herein to describe compounds that are capable of altering the distribution and performance of an active ingredient in the skin and/or exposed tissue, particularly the epidermis, and produce an unusual targeting for underperfused skin and/or pathological tissue in the skin (site of trauma and/or pathology).

The phrase “no penetration modifying agent added” means that the composition comprising a therapeutically active ingredient does not contain any additional ingredient that is defined as a penetration modifying agent.

As used herein, the phrase “essentially free of hyaluronic acid” means that the composition does not contain hyaluronic acid in an amount significant to affect its properties in general; and the transport of the diclofenac, the NSAID or a pharmaceutically acceptable salt thereof.

“Essentially free of hyaluronic acid in a molecular weight of between 150,000 to 750,000 Daltons” means that the composition will not contain significant amounts of hyaluronic acid of molecular weight between 150,000 Daltons and 750,000 Daltons.

As used herein, the phrase “skin disease or disorder”, which is also referred to herein interchangeably as “skin disorder” or “skin condition”, describes a skin condition that is treatable by dermal application, as defined herein, of a drug, herein an NSAID.

As used herein, the phrase “dermal application” describes a topical administration of a drug in which the skin itself is the target organ and is hence typically aimed at localization of the drug in the skin, with little or no systemic absorption of the drug.

NSAIDs in general and diclofenac in particular are known as useful agents for treating skin conditions such as basal cell carcinoma, squamous cell tumors, cutaneous metastatic breast cancer, primary and metastatic melanoma in the skin, malignancies and tumors in the skin, genital warts (condyloma acuminata), psoriasis (both plaque-type psoriasis and nail bed psoriasis), corns on the feet, actinic keratosis, “liver” spots, fungal lesions, and other such types of lesions, and hair loss in pregnancy and hence, preferred skin diseases or disorders according to the present invention include those listed above.

The phrase “pharmaceutically acceptable salt”, as used herein, refers to a charged species of the parent compound and its counter ion, which is typically used to modify the solubility characteristics of the parent compound and/or to reduce any significant irritation to an organism by the parent compound, while not abrogating the biological activity and properties of the administered compound.

An example, without limitation, of a pharmaceutically acceptable salt suitable for use in the context of the present invention is a carboxylate anion of diclofenac or any other NSAID and a cation such as, but not limited to, ammonium, sodium, potassium and the like, preferably sodium.

As used herein the term “about” refers to ±10%. The phrase “weight percentage(s)” or “percent” describes the weight percentage(s) of an ingredient of the total weight of a composition containing the ingredient.

The phrase “greater than” as used herein with respect to a numerical indication (e.g., a concentration, a molecular weight) encompasses any number (integral or factional) that is greater than the indicated number.

The phrase “ranging between” or “ranges between” as used herein with respect to a first numerical indication and a second numerical indication, and the phrase “ranging from” or “ranges from” with respect to a first numerical indication and a second numerical indication, are used interchangeably, and are meant to include the first and second numerical indications, as well as all integral and fractional numerals therebetween.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### DESCRIPTION OF THE PREFERRED EMBODIMENTS

The present invention is of novel compositions, which can be beneficially used for dermal application of an NSAID such as diclofenac and hence can be beneficially used in the treatment of skin conditions such as, but not limited to, basal cell carcinoma, squamous cell tumors, cutaneous metastatic breast cancer, primary and metastatic melanoma in the skin, malignancies and tumors in the skin, genital warts (condyloma acuminata), psoriasis (both plaque-type psoriasis and nail bed psoriasis), corns on the feet, actinic keratosis, “liver” spots, fungal lesions, and other such types of lesions, and hair loss in pregnancy.

The principles and operation of the compositions, and methods according to the present invention may be better understood with reference to the Examples and accompanying description.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present invention provides compositions for the treatment of skin disorders in which dermal application of an NSAID such as diclofenac is beneficial, and which are devoid of the disadvantages of the prior art compositions, particularly those comprising hyaluronic acid, and further particularly those comprising hyaluronic acid in the range of from about 150,000 to about 750,000 Daltons.

While conceiving the present invention, it was envisioned that a composition which is essentially free of hyaluronic acid could be efficiently used in the dermal delivery of an NSAID for treatment of a skin disorder. It was further envisioned that such a composition could be advantageously used when formulated as a gel.

As is demonstrated in the Examples section that follows, while reducing the present invention to practice, it was surprisingly found that compositions comprising a NSAID such as diclofenac or a pharmaceutically acceptable salt thereof, preferably as the sole active ingredient, which are essentially free of hyaluronic acid, more specifically, essentially free of hyaluronic acid having a molecular weight in the range of from about 150,000 to about 750,000 Daltons, have similar or superior therapeutic effects to those of comparable compositions, such as the commercially available preparation Solaraze™, comprising hyaluronic acid having a molecular weight in the range of from about 150,000 to about 750,000 Daltons.

It was surprisingly found that topically applied, dermally penetrating combinations and formulations which employ, combine, or incorporate a therapeutically effective non-toxic amount of diclofenac or a pharmaceutically acceptable salt thereof, and are essentially free of hyaluronic acid, more specifically, essentially free of hyaluronic acid in a molecular weight between 150,000 to 750,000 Daltons, and optionally further comprise other compounds that act as penetration modifying agents, may be used to treat various skin disorders, such as basal cell carcinoma, squamous cell tumors, cutaneous metastatic breast cancer, primary and metastatic melanoma in the skin, malignancies and tumors in the skin, genital warts (condyloma acuminata), psoriasis (both plaque-type psoriasis and nail bed psoriasis), corns on the feet, actinic keratosis, “liver” spots, fungal lesions, and other such types of lesions, and hair loss on the head of a pregnant women, with dramatic success. Furthermore, the preferred compositions and formulations of the invention are systemic independent, quick to penetrate into the skin, particularly to the epidermis and remain there for prolonged periods.

It was also found that commonly used ingredients, which are less expensive and more commercially available than hyaluronic acid, have skin penetration modifying effects similar to those of hyaluronic acid. Examples of such penetration modifying ingredients include urea, diethylene glycol monomethyl ether (““Transcutol”™), glycerine, polysorbate 80 (“Tween”™ 80), or combinations thereof. Hence, it was found that topical compositions, comprising of a NSAID such as diclofenac or a pharmaceutically acceptable salt thereof, preferably as a sole active ingredient, in combination with these ingredients, which are essentially free of hyaluronic acid, have similar or better therapeutically performance than a diclofenac composition comprising hyaluronic acid as a penetration modifying agent. Such compositions have an obvious commercial advantage.

Desired concentrations of the above penetration modifying agents may vary depending on the specific ingredient incorporated into the specific composition, and can be optimized so as to achieve the desired dermal penetration effect while minimizing any undesirable side effect.

Hence, according to one aspect of the present invention, there is provided a pharmaceutical composition which can be beneficially used in the treatment of a skin disease or disorder, as defined hereinabove, and which comprises, as an active ingredient, diclofenac or a pharmaceutically acceptable salt thereof, and a pharmaceutically acceptable carrier and which is essentially free of a hyaluronic acid or a salt thereof which has a molecular weight of between 150,000 and 750,000 Daltons.

While the compositions of the present invention are aimed at dermal delivery of the active ingredient, herein diclofenac, whereby diclofenac is a well known active ingredient usable in the treatment of various skin disorders, conditions that are beneficially treatable by the compositions of the present invention include, without limitation, basal cell carcinoma, squamous cell tumors, cutaneous metastatic breast cancer, primary and metastatic melanoma in the skin, malignancies and tumors in the skin, genital warts (condyloma acuminata), psoriasis (both plaque-type psoriasis and nail bed psoriasis), corns on the feet, actinic keratosis, “liver” spots, fungal lesions, and other such types of lesions, and hair loss on the head of a pregnant women.

However, since NSAIDs other than diclofenac are known as efficient agents in the treatment of such conditions, the compositions described above can optionally comprise any NSAID.

Thus, according to another aspect of the present invention there is provided a pharmaceutical composition as described hereinabove, which comprises, as an active ingredient, a NSAID or a pharmaceutically acceptable salt thereof.

Suitable NSAIDs for use in the context of the present invention include, but are not limited to oxicams, piroxicam, meloxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof, and a combination thereof.

The concentration of the active ingredient in the compositions of the present invention, whether the active ingredient is diclofenac or any other NSAID, preferably ranges between about 0.5 weight percentage and about 5 weight percentages, more preferably between about 0.5 weight percentage and about 4 weight percentages, more preferably between about 0.5 weight percentage and about 3 weight percentages, more preferably between about 1 weight percentage and about 3 weight percentages, more preferably between about 2 weight percentage and about 3 weight percentages, with a presently most preferred concentration being about 3 weight percentages.

As is shown in the Examples section that follows, it was found that compositions that comprise diclofenac alone, as the sole active ingredient, exert a therapeutic effect on skin disorders such as actinic keratosis and hence, in one embodiment of the present invention, the pharmaceutical compositions described above comprise diclofenac, any other NSAID or a salt thereof as the sole active ingredient. Such compositions are highly advantageous in terms of cost, formulation and manufacturing and are further devoid of adverse side effects and adverse drug-drug interactions typically caused when more than one active ingredients are incorporated in a composition.

The use of diclofenac or any NSAID as the sole active ingredient is particularly highly advantageous as compared with prior art NSAID compositions, which, for example, employ, combine, or incorporate, in addition to the NSAID, the known neoplastic agent, alpha-difluoromethylornithine, as an essential active ingredient, since it avoids adverse effects caused by neoplastic agents as well as by interactions with other active ingredients and further renders the composition cost effective and easy to formulate and use.

Hence, in a preferred embodiment of the present invention, the compositions described above are devoid of alpha-difluoromethylornithine.

As is further demonstrated in the Examples section that follows, it was surprisingly found that topically applied dermally penetrating combinations and formulations which employ, combine, or incorporate diclofenac or a pharmaceutically acceptable salt thereof, may be used to treat the skin disorders described above with dramatic success.

It was further surprisingly found that compositions of NSAIDs such as diclofenac or a pharmaceutically acceptable salt thereof with no penetration modifying agent added, have such a desired pharmaceutically performance, thus showing that the incorporation of the disadvantageous hyaluronic acid can be obviated.

As is described hereinabove, it was further surprisingly found that ingredients other than hyaluronic acid of molecular weight between 150,000 and 750,000 Daltons, which are the commonly used, highly available and inexpensive, such as, for example, urea, diethylene glycol monomethyl ether (““Transcutol”™), polysorbate 80, glycerine, hyaluronic acid of molecular weights outside the above indicated range, or combinations thereof.

While, as is described hereinabove, it was found that topically applied dermally penetrating combinations and formulations which employ, combine, or incorporate an NSAID such as diclofenac or a pharmaceutically acceptable salt thereof, and are being essentially free of any penetration modifying agent, may be efficiently used to treat the skin disorders described above with dramatic success, it was also found that commonly used ingredients, which are less expensive and more commercially available than hyaluronic acid, have skin penetration modifying effects similar to those of hyaluronic acid.

Examples of such penetration modifying ingredients include urea, diethylene glycol monomethyl ether (““Transcutol”™), glycerine, polysorbate 80 (“Tween”™ 80), or any combination thereof. Compositions containing such compounds have an obvious commercial advantage over hyaluronic acid-containing preparations.

As is discussed hereinabove, these ingredients are widely used in various topical preparations. However, while most of these ingredients were known heretofore to act as penetration enhancers, it was now surprisingly found that, when incorporated in the compositions of the present invention, these ingredients can be used to achieve the desired dermal drug delivery, allowing the active ingredient (an NSAID such as diclofenac) to penetrate the stratum corneum, remain in the targeted site, preferably without reaching the systemic circulation and allowing for a prolonged stay of the active ingredient in the affected area, and thus act as penetration modifiers.

Thus, in a preferred embodiment of the present invention, the compositions described above further comprise urea. While, as is discussed in detail hereinabove, urea is oftentimes used in topically applied compositions as a penetration enhancer, the present inventors have surprisingly found that when incorporated in the compositions of the present invention, urea acts as a penetration modifier.

More specifically, it was found that urea, in a concentration of between 5 weight percentages and about 15 weight percentages, more preferably between about 5 weight percentages and about 10 weight percentages, modifies and delays the skin penetration of an NSAID, such as diclofenac or a pharmaceutically acceptable silt thereof maintaining the drug in the skin for a prolonged time, preferably without reaching the systemic circulation.

While compositions which comprise urea are oftentimes associated with adverse characteristics due to the instability and thus decomposition of urea, compositions according to the present invention which utilize urea as a penetration modifier preferably further comprise a substance that is capable of stabilizing the urea. Stabilization of urea may be achieved, for example, by maintaining the desired pH or affecting other chemical and physical properties of the formulation and hence these substances may be, for example, an alpha hydroxy acid, a beta hydroxyacid, allantoin, hydrochloric acid, a buffer system, an antioxidant, or any mixture thereof.

In another preferred embodiment, the compositions described above comprise diethylene glycol monomethyl ether, also known by its trade name Transcutol™.

As is shown in the Examples section that follows, it was found that Transcutol™ can be used for the dermal delivery of a drug, maintaining the drug in the skin for a prolonged time, preferably without reaching the systemic circulation. It was further found that an advantageous relatively low concentration of Transcutol™ is useful for the dermal delivery of an NSAID such as diclofenac or a pharmaceutically acceptable salt thereof, preferably between about 5 weight percentages and about 15 weight percentages, and more preferably between about 5 weight percentages and about 10 weight percentages. Such a relatively low concentration sands in line with, for example, the FDA regulations, according to which the maximal concentration of Transcutul™ that can be used in topical preparations is 10 weight percentages, and is therefore advantageous over the prior art formulations described above, in which Transcutul™ in a concentration of up to 50 weight percentages is used.

In another preferred embodiment, the compositions described above comprise glycerine. As is shown in the Examples section that follows, it was also found that glycerine, a known penetration enhancer, at a concentration of from about 20 weight percentages and about 50 weight percentages, more preferably at a concentration of from about 30 weight percentages and about 50 weight percentages is useful as a penetration modifier for dermal delivery of an NSAID.

It was fierier found that combinations of glycerine in concentrations between about 20 weight percentages and about 50 weight percentages, preferably between about 20 weight percentages and about 40 weight percentages, together with diethylene glycol monomethyl ether (Transcutol™) in concentrations of from about 5% to about 15% by weight improve the retention time of the drug in the skin relative to the action of each penetration modulator alone, and hence, in yet another preferred embodiment, the compositions described above comprise such a combination of glycerine and diethylene glycol monomethyl ether.

It was further found that Polysorbate 80 (also known as TWEEN 80) can be efficiently used as a penetration modifier, preferably at a concentration that ranges between about 1 weight percentage and about 5 weight percentages, more preferably between about 2 weight percentages and about 3 weight percentages, and thus, in another preferred embodiment, the compositions described above further comprise Polysorbate 80, preferably, at a concentration within the concentration ranges indicated above.

Hyaluronic acid in a molecular weight outside the range of 150,000 Daltons to 750,000 Daltons may also be used in a concentration range of about 1% to 3% by weight, more preferably about 2.5% by weight, according to another preferred embodiment of the present invention.

The preferred concentrations of the various penetration modifiers that can be incorporated in the compositions of the present invention are generally determined as those which are sufficient to facilitate the drug's dermal penetration to the site in the skin to be treated, through the tissue (including any scar tissue) or through the cell membrane.

Further according to the present invention, each of the compositions described above further comprises a pharmaceutically acceptable carrier.

Examples of pharmaceutically acceptable carriers that are usable in the context of the present invention include carrier materials that are well-known for use in the medical arts as bases for e.g., emulsions, creams, aqueous solutions, oils, ointments, pastes, gels, lotions, milks, foams, suspensions, acrosols, patches and the like, depending on the final form of the composition.

Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions.

According to a preferred embodiment of the present invention, the carrier comprises water and a polyalkylene glycol such as, for example, methoxypolyethylene glycol having a MW of between 350 kD (MPEG 350) and 550 kD (MPEG 550).

By selecting the appropriate carrier and optionally other ingredients that can be included in the composition, as is detailed hereinbelow, the composition of the present invention may be formulated into any form normally employed for topical application. Hence, the composition of the present invention can be, for example, in a form of a cream, an ointment, a paste, a gel, a lotion, a milk, a suspension, an aerosol, a spray, a foam a serum, a swab, a pledget, a pad and a patch.

It will be appreciated that the final form of a topical composition plays an important role in its efficacy and its usage convenience.

In a preferred embodiment of the present invention, the composition is formulated as a gel. As is well known in the art, gel formulations are easy to apply, without being too runny, greasy, or otherwise inconvenient to use by the patient, and are therefore highly advantageous in topical applications in general and dermal applications in particular.

When formulated as a gel, each of the compositions of the present invention further comprises one or more gelling agents, such as, but not limited to hydroxyethyl cellulose or hydroxypropylmethyl cellulose. However, any other pharmaceutically acceptable thickening/gelling agent may be used, such as other cellulosic ethers, polymeric thickening agents such as xanthan gum, guar gum, and the like, fatty alcohols, fatty acids and their alkali salts and mixtures thereof, as well as inorganic thickeners/gelling agents.

The amount of gelling agent is not particularly critical, and can be selected to provide the desired product consistency or viscosity to allow for easy application to the skin, but which will not be too watery or loose. Generally, depending upon its molecular weight, amounts of thickening agent of from about 0.1 weight percentage to about 7 weight percentages, preferably from about 0.1 weight percentage to about 5 weight percentages, more preferably from about 1 weight percentage to about 3 weight percentages, of the total weight of the composition will provide the desired effect.

The compositions of the present invention can optionally further comprise a variety of components that are suitable for rendering the composition more aesthetically acceptable or to provide the composition with additional usage benefits. Such conventional optional components or ingredients are well known to those skilled in the art and are referred to herein as “additives”. Some non-limiting representative examples of these ingredients include humectants, deodorants, antiperspirants, sun screening agents, sunless tanning agents, hair conditioning agents, pH adjusting agents, chelating agents, preservatives, emulsifiers, occlusive agents, emollients, thickeners, solubilizing agents, penetration enhancers, anti-irritants, colorants and surfactants.

The compositions of the present invention may therefore further include one or more additives. The concentration of the additive(s) preferably ranges between about 1 weight percentage and about 5 weight percentages.

Representative examples of emollients that are suitable for use in the compositions of the present invention include., without limitation, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Representative examples of solubilizing agents that are suitable for use in the compositions of the present invention include, without limitation, citric acid, ethylenediaminetetraacetate, sodium meta-phosphate, succinic acid, urea, cyclodextrin, polyvinylpyrolidone, diethylammonium-ortho-benzoate, micelle-forming solubilizers, polyoxyethylene sorbitan fatty acid ester, polyoxyethylene n-alkyl ethers, n-alkyl amine n-oxides, poloxamers, organic solvents, phospholipids and cyclodextrines.

Representative examples of deodorant agents that are usable in the context of the present invention include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmithyl sarcosine, lauroyl sarcosine, N-myristoyl glycine, potassium N-lauryl sarcosine, stearyl trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Suitable preservatives that can be used in the context of the present composition include, without limitation, one or more alkanols, aryl alcohols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or any combinations thereof.

Suitable emulsifiers that can be used in the context of the present invention include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents that can be used in the context of the present invention include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

In a preferred embodiment of the present invention, the compositions described above comprise a preservative such as benzyl alcohol.

The compositions of the present invention may be packed or presented in any convenient way. For example, they may be packed in a tube, a bottle, a unit dosage form or a pressurized container, using techniques well known to those skilled in the art and as set forth in reference works such as Remington's Pharmaceutical Science 15^{th} Ed. It is preferred that the packaging is done in such a way so as to minimize contact of the unused compositions with the environment, in order to minima contamination of the compositions before and after the container is opened.

As is demonstrated in the Examples section that follows, the composition of the present invention can be efficiently used for treating skin conditions in which dermal application of an NSAID such as diclofenac is beneficial, as is detailed hereinabove.

Thus, in a preferred embodiment of the present invention, each of the compositions described hereinabove is packaged in a packaging material and is identified in print, in or on the package, for use in the treatment of such a skin condition. Examples of such skin conditions include, without limitation, skin conditions such as basal cell carcinoma, squamous cell tumors, cutaneous metastatic breast cancer, primary and metastatic melanoma in the skin, malignancies and tumors in the skin, genital warts (condyloma acuminata), psoriasis (both plaque-type psoriasis and nail bed psoriasis), corns on the feet, actinic keratosis, “liver” spots, fungal lesions, and other such types of lesions, as well as hair loss in pregnancy.

According to another aspect of the invention there is provided a method of treating skin disorders, as is described hereinabove. The method is effected by applying onto one or more biological surfaces of a subject, which are affected by the skin disorder, a therapeutically effective amount, as is defined hereinabove, of any one of the compositions described hereinabove.

The above methods may employ the use of the any of compositions of the present invention or combinations thereof by applying the composition or combination a number of times daily (for a certain period of time. Thus, for example, the composition of combination can be applied between 1 and 4 times a day, preferably between once and twice a day, for 20-120 days, preferably 30-60 days.

As is demonstrated in the Examples section that follows, an exemplary composition according to the present invention was found highly active in treating actinic keratosis, when applied twice daily, for a period of 60 days.

The present invention further encompasses processes for the preparation of the pharmaceutical compositions described above. These processes generally comprise admixing the active ingredients described hereinabove and the pharmaceutically acceptable carrier. In cases where other agents or active agents, as is detailed hereinabove, are present in the compositions, the process includes admixing these agents together with the active ingredients and the carrier. A variety of exemplary formulation techniques that are usable in the process of the present invention is described, for example, in Harry's Cosmeticology, Seventh Edition, Edited by J B Wilkinson and R J Moore, Longmann Scientific & Technical, 1982, Chapter 13 “The Manufacture of Cosmetics” pages 757-799.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

The following examples further describe and demonstrate embodiments within the scope of the subject invention. In said examples to follow, all parts and percentages are given by weights. The examples are given solely for the purpose of illustration and are not to be constructed as limitations of the subject invention, as many variations thereof are possible without departing from the spirit and scope of the invention.

### Example 1

### Topical Diclofenac Sodium Gel Composition with No Penetration Modifying Agent

A representative example of a 3% diclofenac sodium gel formulation according to the present invention, free of hyaluronic acid and any other penetration modifier, described in Table 1, was prepared. The formulation includes benzyl alcohol as a preservative, methoxy polyethylene glycol 350 (MPEG 350) and water as the pharmaceutically acceptable carrier and Methocel™ (HPMC) as gelling agent.

TABLE 1

| Ingredient | Concentration % w/w |
|---|---|
| Diclofenac sodium | 3.00 |
| Benzyl alcohol | 1.00 |
| MPEG 350 | 20.00 |
| Methocel ™ | 2.80 |
| HCl 0.1N | 0.15 |
| Purified water | 73.05 |

A preliminary test of the efficacy of his formulation Solaraze™ in the treatment of actinic keratosis was conducted in vitro on an artificial membrane.

The results, presented in Table 2, showed that a composition according to the present invention, having no penetration modifier added, has dermal penetration characteristics sufficient to exert the desired pharmacological effect.

TABLE 2

Penetration

| | Mean penetrated amount | |
|---|---|---|
| Time | (mcg) | (Standard deviation) |
| 0 | 0 | 0 |
| 1 | 0.501056 | 0.0491 |
| 2 | 1.717283 | 0.058972 |
| 4 | 3.102223 | 0.085956 |
| 5 | 3.356 | 0.085381 |
| 7 | 3.535081 | 0.105439 |
| 16 | 4.132458 | 0.090286 |
| 18 | 4.317976 | 0.065684 |
| 20 | 4.454235 | 0.039047 |
| 22 | 4.55817 | 0.039142 |
| 24 | 4.658521 | 0.064727 |

### Example 2

### Topical Diclofenac Sodium Gel Composition with Diethylene Glycol Monomethyl Ether as a Penetration Modifier

An exemplary 3% diclofenac sodium gel formulation according to the present invention, free of hyaluronic acid, and containing 10% diethylene glycol monomethyl ether (“Transcutol”™) as a penetration modifier, described in Table 3, was prepared.

TABLE 3

| Ingredient | Concentration % w/w |
|---|---|
| Diclofenac sodium | 3.00 |
| Benzyl alcohol | 1.00 |
| MPEG 350 | 20.00 |
| Methocel ™ | 2.20 |
| “Transcutol” ™ | 10.00 |
| Purified water | 63.80 |

### Example 3

### Topical Diclofenac Sodium Get Composition with Glycerine as a Penetration Modifier

An exemplary 3% diclofenac sodium gel formulation according to the present invention, free of hyaluronic acid and containing 40% glycerine as a penetration modifier, described in Table 4, was prepared.

TABLE 4

| Ingredient | Concentration % w/w |
|---|---|
| Diclofenac sodium | 3.00 |
| Benzyl alcohol | 1.00 |
| MPEG 350 | 20.00 |
| Natrosol ™ 250 HX | 1.50 |
| Glycerine | 40.00 |
| Purified water | 34.50 |

### Examples 4-8

### Topical Diclofenac Sodium Gel Compositions with Urea as a Penetration Modifier

Several exemplary 3% diclofenac sodium gel formulations according to the present invention, free of hyaluronic acid and containing different concentrations of urea as a penetration modifier, as described in Table 5, were prepared. In some of the examples allantoin was added as a stabilizer component. In some of the examples one or more pH stabilizing components were added, either buffer solution (e.g. citric acid with sodium phosphate dibasic), or HCl.

TABLE 5

| | Concentration % w/w | | | | |
|---|---|---|---|---|---|
| Ingredient | Example 4 | Example 5 | Example 6 | Example 7 | Example 8 |
| Diclofenac | 3.0 | 3.0 | 3.0 | 3.0 | 3.0 |
| sodium | | | | | |
| Benzyl | 1.0 | 1.0 | 1.0 | 1.0 | 1.0 |
| alcohol | | | | | |
| MPEG 350 | 20.0 | 20.0 | 20.0 | | |
| MPEG 550 | | | | 20.0 | 20.0 |
| Urea | 7.0 | 7.0 | 7.0 | 10.0 | 4.0 |
| Allantoin | | | | 0.5 | 0.2 |
| Buffer | | 60.0 | | 20.0 | 20.0 |
| solution* | | | | | |
| Methocel ™ | 2.5 | 2.5 | 2.5 | 2.5 | 2.5 |
| HCl | | | 0.2 | | |
| P. Water | 66.5 | 6.5 | 66.3 | 43.0 | 49.3 |

*(citric acid + sodium phosphate dibasic)

### Example 9

### Topical Diclofenac Sodium Gel with Polysorbate 80 as a Penetration Modifier

An exemplary 3% diclofenac sodium gel formulation according to the present invention, free of hyaluronic acid and containing 3% polysorbate 80 (“Tween 80”™) as a penetration modifier, described in Table 6, was prepared.

TABLE 6

| Ingredient | Concentration % w/w |
|---|---|
| Diclofenac sodium | 3.0 |
| Benzyl alcohol | 1.0 |
| MPEG 350 | 20.0 |
| Methocel ™ | 2.0 |
| Polysorbate 80 (“Tween 80”) | 3.0 |
| Purified water | 71.0 |

### Examples 10-11

### Topical Diclofenac Sodium Gel Compositions with Hyaluronic Acid in a Molecular Weight Outside the 150,000 to 750,000 Daltons Range.

Two 3% diclofenac sodium gel formulations according to the present invention, containing 2.5% hyaluronic acid as a penetration modifier, as described in Table 7, were prepared. The hyaluronic acid in example 10 is of a molecular weight of 100,000 Daltons, while the hyaluronic acid in example 11 is of a molecular weight of 900,000 Daltons.

TABLE 7

Concentration % w/w

| Ingredient | Example 10 | Example 11 |
|---|---|---|
| Diclofenac sodium | 3.0 | 3.0 |
| Benzyl alcohol | 1.0 | 1.0 |
| MPEG 350 | 20.0 | 20.0 |
| Hyaluronic acid - molecular | 2.5 | |
| weight of 100,000 Daltons | | |
| Hyaluronic acid - molecular | | 2.5 |
| weight of 900,000 Daltons | | |
| Purified water | 73.5 | 73.5 |

### Example 12

### Topical Diclofenac Sodium Get Composition with a Combination of Glycerine and “Transcutol” as a Penetration Modifier

An exemplary 3% diclofenac sodium gel formulation according to the present invention, free of hyaluronic acid and containing 30% glycerine and 10% “Transcutol” as penetration modifiers, as described in Table 8, was prepared.

TABLE 8

| Ingredient | Concentration % w/w |
|---|---|
| Diclofenac sodium | 3.00 |
| Benzyl alcohol | 1.00 |
| MPEG 350 | 20.00 |
| Natrosol ™ 250 HX | 1.50 |
| Glycerine | 40.00 |
| “Transcutol” ™ | 10.00 |
| Purified water | 24.50 |

### Example 13

Comparative tests of the efficacy of exemplary formulation according to the present invention and the commercially available product Solaraze™ in the treatment of actinic keratosis was conducted in vitro on both artificial membrane and human skin.

Thus, the efficacy of the compositions described above in Examples 2 and 6 was compared with that of Solaraze™ in an in vitro test conducted on an artificial membrane. The results, presented in Table 9, showed that the compositions of the present invention exert at least similar if not better activity as Solaraze™.

TABLE 9

| | Penetrated Amount | | | | | |
|---|---|---|---|---|---|---|
| | (mcg) | | | | | |
| | Solaraze ™ | | Urea formulation | | Transcutul formulation | |
| Time | Mean | | Mean | | Mean | |
| (h) | amount | SD | amount | SD | amount | SD |
| 0 | 0 | 0 | 0 | 0 | 0 | 0 |
| 1 | 0.218 | 0.035777 | 0.3 | 7.07E−05 | 0.458276 | 0.083972 |
| 2 | 0.642 | 0.029768 | 0.69 | 1E−04 | 0.971782 | 0.101306 |
| 4 | 1.190583 | 0.019978 | 1.25 | 1E−04 | 1.397062 | 0.186669 |
| 5 | 1.391051 | 0.03985 | 1.36 | 1E−04 | 1.45995 | 0.171645 |
| 7 | 1.533942 | 0.059605 | 1.95 | 1E−04 | 1.677308 | 0.11767 |
| 16 | 1.760775 | 0.097401 | 1.95 | 1E−04 | 2.347669 | 0.126389 |
| 18 | 1.77686 | 0.124672 | 2 | 1E−04 | 2.481913 | 0.135006 |
| 20 | 1.797274 | 0.166598 | 2.04 | 1E−04 | 2.569507 | 0.152388 |
| 22 | 1.845208 | 0.157036 | 2.159999 | 1E−04 | 2.683668 | 0.125479 |
| 24 | 1.890345 | 0.165446 | 2.19 | 1E−04 | 2.823991 | 0.14343 |

The efficacy of the compositions described above in Examples 2 and 3 was compared with that of Solaraze™ in an in vitro test conducted on human skin. The results, presented in Table 10, showed that the compositions of the present invention exert at least similar if not better activity as Solaraze™.

TABLE 10

| | Penetrated Amount | | | | | |
|---|---|---|---|---|---|---|
| | (mcg) | | | | | |
| | Solaraze ™ | | Glycerine formulation | | Transcutul formulation | |
| Time | Mean | | Mean | | Mean | |
| (h) | amount | SD | amount | SD | amount | SD |
| 0 | 0 | 0 | 0 | 0 | 0 | 0 |
| 1 | 0.18 | 0.08 | 0.06 | 0.02 | 0.15 | 0.06 |
| 2 | 0.23 | 0.05 | 0.05 | 0 | 0.26 | 0.04 |
| 4 | 0.37 | 0.03 | 0.1 | 0.03 | 0.48 | 0.06 |
| 5 | 0.38 | 0.03 | 0.1 | 0.04 | 0.53 | 0.08 |
| 7 | 0.45 | 0.04 | 0.12 | 0.05 | 0.64 | 0.12 |
| 16 | 0.55 | 0.06 | 0.2 | 0.1 | 0.78 | 0.16 |
| 18 | 0.59 | 0.07 | 0.22 | 0.12 | 0.82 | 0.17 |
| 20 | 0.6 | 0.09 | 0.24 | 0.13 | 0.89 | 0.19 |
| 22 | 0.66 | 0.11 | 0.25 | 0.12 | 0.9 | 0.19 |
| 24 | 0.66 | 0.1 | 0.14 | 0.03 | 0.94 | 0.21 |

### Example 14

### Results of a Small-Scale Clinical Study Performed with the Topical Diclofenac Sodium Gel Composition of Example 2

A single center, double-blind, pilot study that included 80 patients was conducted in order to compare the formulation according to the present invention described in Example 2 (hereinafter, Composition 2) and a hyaluronate-containing commercially available product (Solaraze™).

The aims of the study were to compare the efficacy and safety of the two 3% diclofenac-containing gel formulations. Patients were treated with either Composition 2 of Solaraze™ according to the assigned treatment group. The medication was applied twice daily for a period of 60 days. Efficacy and safety were assessed in each of four visits: at time 0, after 30 days, after 60 days, and at a follow-up visit 30 days after the end of the treatment. Assessment was based upon three indices: Investigators and patient's global improvement indices, lesion total thickness score and total lesion number count.

Table 11 below presents the investigator's evaluation of the improvement in the lesions severity following treatment with the two formulations. The values are compared to the baseline unit, observed in visit 1.

According to the results of this study, the two products were found to be equally safe and effective in the treatment of actinic keratosis. No statistically significant difference was found between the drugs.

TABLE 11
(severity score)

| Drug | Visit 2 | Visit 3 | Visit 4 |
|---|---|---|---|
| Composition 2 | 45.8572 | 67.1738 | 61.8215 |
| Commercial | 48.1237 | 68.0938 | 67.9055 |
| product | | | |
| P value | 0.7375 | 0.9622 | 0.2745 |

1. A pharmaceutical composition, identified for use in the treatment of a skin disease or disorder, comprising, as a sole active ingredient, diclofenac or a pharmaceutically acceptable salt thereof, and a pharmaceutically acceptable carrier, the composition being essentially free of hyaluronic acid or a salt thereof having a molecular weight of between 150,000 and 750,000 Daltons.

2. The pharmaceutical composition of claim 1, wherein said skin disease or disorder is selected from the group consisting of basal cell carcinoma, squamous cell tumor, cutaneous metastatic breast cancer, pima and metastatic melanoma in the skin, malignancies and tumors in the skin, genital warts, psoriasis, corns on the feet, actinic keratosis, liver spots, skin lesions, fungal lesions, and hair loss in pregnancy.

3. The pharmaceutical composition of claim 2, wherein said skin disease or disorder is actinic keratosis.

4. The pharmaceutical composition of claim 1, further comprising at least one penetration modifier.

5. The pharmaceutical composition of claim 4, wherein said penetration modifier is selected from the group consisting of diethylene glycol monomethyl ether, urea, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof.

6. The pharmaceutical composition of claim 4, wherein said at least one penetration modifier comprises diethylene glycol monoethyl ether.

7. The pharmaceutical composition of claim 6, wherein a concentration of said diethylene glycol monoethyl ether ranges between about 5 weight percentages and about 15 weight percentages of the total weight of the composition.

8. The pharmaceutical composition of claim 7, wherein said concentration of said diethylene glycol monoethyl ether ranges between about 5 weight percentages and about 10 weight percentages.

9. The pharmaceutical composition of claim 4, wherein said at least one penetration modifier comprises urea.

10. The pharmaceutical composition of claim 9, wherein a concentration of said urea ranges between about 5 weight percentages and about 15 weight percentages of the total weight of the composition.

11. The pharmaceutical composition of claim 10, wherein said concentration of said urea ranges between about 5 weight percentages and about 10 weight percentages.

12. The pharmaceutical composition of claim 9, further comprising at least one pH-stabilizing agent.

13. The pharmaceutical composition of claim 12, wherein said at least one pH stabilizing agent is selected from the group consisting of a hydroxyacid, allantoin, hydrochloric acid, a buffer system, an antioxidant and any mixture thereof.

14. The pharmaceutical composition of claim 4, wherein said at least one penetration modifier comprises glycerine.

15. The pharmaceutical composition of claim 14, wherein a concentration of said glycerine ranges between about 20 weight percentages and about 50 weight percentages of the total weight of the composition.

16. The pharmaceutical composition of claim 14, wherein said at least one penetration modifier further comprises diethylene glycol monoethyl ether.

17. The pharmaceutical composition of claim 16, wherein a concentration of said diethylene glycol monoethyl ether is about 10 weight percentages.

18. The pharmaceutical composition of claim 16, wherein a concentration of said glycerine ranges between about 20 weight percentages and about 40 weight percentages, and wherein a concentration of said diethylene glycol monoethyl ether ranges between about 5 weight percentages and about 15 weight percentages.

19. The pharmaceutical composition of claim 4, wherein said at least one penetration modifier comprises polysorbate 80.

20. The pharmaceutical composition of claim 19, wherein a concentration of said polysorbate 80 ranges between about 1 weight percentage and about 5 weight percentages of the total weight of the composition.

21. The pharmaceutical composition of claim 20, wherein said concentration of said polysorbate ranges between about 2 weight percentages and about 3 weight percentages.

22. The pharmaceutical composition of claim 4, wherein said at least one penetration modifier is a hyaluronic acid selected from the group consisting of a hyaluronic acid or a salt thereof having a molecular weight of less than about 150,000 and a hyaluronic acid or a salt thereof having a molecular weight greater than about 750,000.

23. The pharmaceutical composition of claim 22, wherein a concentration of said hyaluronic acid ranges between about 1 weight percentage and about 3 weight percentages of the total weight of the composition.

24. The pharmaceutical composition of claim 23, wherein said concentration of said hyaluronic acid is about 2.5 weight percentages.

25. The pharmaceutical composition of claim 1, wherein said pharmaceutically acceptable salt is a sodium salt.

26. The pharmaceutical composition of claim 1, wherein a concentration of said diclofenac or said pharmaceutically acceptable salt thereof ranges between about 1 weight percentage and about 5 weight percentages of the total weight of the composition.

27. The pharmaceutical composition of claim 26, wherein a concentration of said diclofenac or said pharmaceutically acceptable salt thereof is about 3 weight percentages.

28. The pharmaceutical composition of claim 1, being formulated in a form selected from the group consisting of a gel, a cream, an ointment, a paste, a lotion, a milk, a suspension, an aerosol, a spray, a foam, a serum, a swab, a pledglet, a pad and a patch.

29. The pharmaceutical composition of claim 28, being formulated in the form of a gel.

30. The pharmaceutical composition of claim 29, further comprising a gelling agent.

31. The pharmaceutical composition of claim 30, wherein said gelling agent is selected from the group consisting of hydroxypropyl methylcellulose and hydroxethylcellulose.

32. The pharmaceutical composition of claim 30, wherein a concentration of said gelling agent ranges between about 0.1 weight percentage and about 7 weight percentages of the total weight of the composition.

33. The pharmaceutical composition of claim 32, wherein a concentration of said gelling agent ranges between about 1 weight percentage and about 3 weight percentages.

34. The pharmaceutical composition of claim 1 further comprising at least one additive.

35. The pharmaceutical composition of claim 34, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

36. The pharmaceutical composition of claim 34, wherein a concentration of said additive ranges between about 1 weight percentage and about 5 weight percentages of the total weight of the composition.

37. The pharmaceutical composition of claim 1, packaged in a packaging material and identified in print, in or on the package, for use in the treatment of said skin disease or disorder.

38. The pharmaceutical composition of claim 37, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

39. A method for treating a skin disease or disorder in a subject in need thereof, the method comprising applying onto at least one biological surface affected by said skin disease or disorder of said subject a therapeutically effective amount of the pharmaceutical composition of claim 1.

40. The method of claim 39, wherein said at least one biological surface is selected from the group consisting of the skin of the face, an ear, a scalp, a neck, a forearm, a back, a leg, an arms and a hand.

41. The method of claim 40, wherein said applying is performed between 1 and 4 times a day, for a time period that ranges between 20 and 120 days.

42. The method of claim 41, wherein said applying is performed twice a day.

43. The method of claim 42, wherein said time period ranges between 30 and 60 days.

44. The method of claim 39, wherein said subject is a human.

45. The method of claim 39, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

46. The method of claim 45, wherein said skin disease or disorder is actinic keratosis.

47. The method of claim 39, wherein said pharmaceutical composition further comprises at least one penetration modifier.

48. The method of claim 47, wherein said penetration modifier is selected from the group consisting of diethylene glycol monomethyl ether, urea, glycerine, polysorbate 80, a hyaluronic acid or a salt thereof having a molecular weight less than about 150,000 Daltons, a hyaluronic acid or a salt thereof having a molecular weight greater than about 750,000 Daltons, and any mixture thereof.

49. The method of claim 48, wherein said at least one penetration modifier comprises diethylene glycol monoethyl ether.

50. The method of claim 49, wherein a concentration of said diethylene glycol monoethyl ether ranges between about 5 weight percentages and about 15 weight percentages of the total weight of said composition.

51. The method of claim 50, wherein said concentration of said diethylene glycol monoethyl ether ranges between about 5 weight percentages and about 10 weight percentages.

52. The method of claim 48, wherein said at least one penetration modifier comprises urea.

53. The method of claim 52, wherein a concentration of said urea ranges between about 5 weight percentages and about 15 weight percentages of the total weight of said composition.

54. The method of claim 53, wherein said concentration of said urea ranges between about 5 weight percentages and about 10 weight percentages.

55. The method of claim 48, wherein said composition further comprises at least one pH-stabilizing agent.

56. The method of claim 55, wherein said at least one pH stabilizing agent is selected from the group consisting of a hydroxyacid, allantoin, hydrochloric acid, a buffer system, an antioxidant and any mixture thereof.

57. The method of claim 47, wherein said at least one penetration modifier comprises glycerine.

58. The method of claim 57, wherein a concentration of said glycerine ranges between about 20 weight percentages and about 50 weight percentages of the total weight of said composition.

59. The method of claim 57, wherein said at least one penetration modifier further comprises diethylene glycol monoethyl ether.

60. The method of claim 59, wherein a concentration of said diethylene glycol monoethyl ether is about 10 weight percentages of the total weight of said composition.

61. The method of claim 59, wherein a concentration of said glycerine ranges between about 20 weight percentages and about 40 weight percentages, and wherein a concentration of said diethylene glycol monoethyl ether ranges between about 5 weight percentages and about 15 weight percentages.

62. The method of claim 47, wherein said at least one penetration modifier comprises polysorbate 80.

63. The method of claim 62, wherein a concentration of said polysorbate 80 ranges between about 1 weight percentage and about 5 weight percentages of the total weight of said composition.

64. The method of claim 63, wherein said concentration of said polysorbate ranges between about 2 weight percentages and about 3 weight percentages.

65. The method of claim 47 wherein said at least one penetration modifier is hyaluronic acid selected from the group consisting of a hyaluronic acid or a salt thereof having a molecular weight less than about 150,000 and a hyaluronic acid or a salt thereof having a molecular weight greater than about 750,000.

66. The method of claim 65, wherein a concentration of said hyaluronic acid ranges between about 1 weight percentage and about 3 weight percentages of the total weight of said composition.

67. The method of claim 66, wherein said concentration of said hyaluronic acid is about 2.5 weight percentages.

68. The method of claim 39, wherein said pharmaceutically acceptable salt is a sodium salt.

69. The method of claim 39, wherein a concentration of said diclofenac or said pharmaceutically acceptable salt ranges between about 1 weight percentage and about 5 weight percentages of the total weight of said composition.

70. The method of claim 69, wherein a concentration of said diclofenac or said pharmaceutically acceptable salt thereof is about 3 weight percentages.

71. The method of claim 39, wherein said composition is formulated in a form selected from the group consisting of a gel, a cream, an ointment, a paste, a lotion, a milk, a suspension, an aerosol, a spray, a foam, a serum, a swab, a pledglet, a pad and a patch.

72. The method of claim 71, wherein said composition is formulated in the form of a gel.

73. The method of claim 72, wherein said composition further comprises a gelling agent.

74. The method of claim 73, wherein said gelling agent is selected from the group consisting of hydroxypropyl methylcellulose and hydroxethylcellulose.

75. The method of claim 73, wherein a concentration of said gelling agent ranges between about 0.1 weight percentage and about 7 weight percentages of the total weight of the composition.

76. The method of claim 74, wherein a concentration of said gelling agent ranges between about 1 weight percentage and about 3 weight percentages.

77. The method of claim 39, wherein said composition further comprises at least one additive.

78. The method of claim 77, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

79. The method of claim 78, wherein a concentration of said additive ranges between about 1 weight percentage and about 5 weight percentages.

80. A pharmaceutical composition, identified for use in the treatment of a skin disease or disorder, comprising, as an active ingredient, diclofenac or a pharmaceutically acceptable salt thereof, and a pharmaceutically acceptable carrier, the composition being essentially free of hyaluronic acid or a salt thereof having a molecular weight of between 150,000 and 750,000 Daltons and is further being devoid of alpha-difluoromethylornithine.

81. The pharmaceutical composition of claim 80, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

82. The pharmaceutical composition of claim 81, wherein said skin disease or disorder is actinic keratosis.

83. The pharmaceutical composition of claim 80, further comprising at least one penetration modifier.

84. The pharmaceutical composition of claim 83, wherein said penetration modifier is selected from the group consisting of diethylene glycol monomethyl ether, urea, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof.

85. The pharmaceutical composition of claim 83, wherein said at least one penetration modifier comprises diethylene glycol monoethyl ether.

86. The pharmaceutical composition of claim 85, wherein a concentration of said diethylene glycol monoethyl ether ranges between about 5 weight percentages and about 15 weight percentages of the total weight of the composition.

87. The pharmaceutical composition of claim 86, wherein said concentration of said diethylene glycol monoethyl ether ranges between about 5 weight percentages and about 10 weight percentages.

88. The pharmaceutical composition of claim 83, wherein said at least one penetration modifier comprises urea.

89. The pharmaceutical composition of claim 88, wherein a concentration of said urea ranges between about 5 weight percentages and about 15 weight percentages of the total weight of the composition.

90. The pharmaceutical composition of claim 89, wherein said concentration of said urea ranges between about 5 weight percentages and about 10 weight percentages.

91. The pharmaceutical composition of claim 88, further comprising at least one pH-stabilizing agent.

92. The pharmaceutical composition of claim 91, wherein said at least one pH stabilizing agent is selected from the group consisting of a hydroxyacid, allantoin, hydrochloric acid, a buffer system, an antioxidant and any mixture thereof.

93. The pharmaceutical composition of claim 83, wherein said at least one penetration modifier comprises glycerine.

94. The pharmaceutical composition of claim 93, wherein a concentration of said glycerine ranges between about 20 weight percentages and about 50 weight percentages of the total weight of the composition.

95. The pharmaceutical composition of claim 93, wherein said at least one penetration modifier further comprises diethylene glycol monoethyl ether.

96. The pharmaceutical composition of claim 95, wherein a concentration of said diethylene glycol monoethyl ether is about 10 weight percentages.

97. The pharmaceutical composition of claim 95, wherein a concentration of said glycerine ranges between about 20 weight percentages and about 40 weight percentages, and wherein a concentration of said diethylene glycol monoethyl ether ranges between about 5 weight percentages and about 15 weight percentages.

98. The pharmaceutical composition of claim 83, wherein said at least one penetration modifier comprises polysorbate 80.

99. The pharmaceutical composition of claim 98, wherein a concentration of said polysorbate 80 ranges between about 1 weight percentage and about 5 weight percentages of the total weight of the composition.

100. The pharmaceutical composition of claim 99, wherein said concentration of said polysorbate ranges between about 2 weight percentages and about 3 weight percentages.

101. The pharmaceutical composition of claim 83, wherein said at least one penetration modifier is a hyaluronic acid selected from the group consisting of a hyaluronic acid or a salt thereof having a molecular weight of less than about 150,000 and a hyaluronic acid or a salt thereof having a molecular weight greater than about 750,000.

102. The pharmaceutical composition of claim 101, wherein a concentration of said hyaluronic acid ranges between about 1 weight percentage and about 3 weight percentages of the total weight of the composition.

103. The pharmaceutical composition of claim 102, wherein said concentration of said hyaluronic acid is about 2.5 weight percentages.

104. The pharmaceutical composition of claim 80, wherein said pharmaceutically acceptable salt is a sodium salt.

105. The pharmaceutical composition of claim 80, wherein a concentration of said diclofenac or said pharmaceutically acceptable salt thereof ranges between about 1 weight percentage and about 5 weight percentages of the total weight of the composition.

106. The pharmaceutical composition of claim 105, wherein a concentration of said diclofenac or said pharmaceutically acceptable salt thereof is about 3 weight percentages.

107. The pharmaceutical composition of claim 80, being formulated in a form selected from the group consisting of a gel, a cream, an ointment, a paste, a lotion, a milk a suspension, an aerosol, a spray, a foam, a serum, a swab, a pledglet, a pad and a patch.

108. The pharmaceutical composition of claim 107, being formulated in the form of a gel.

109. The pharmaceutical composition of claim 108, further comprising a gelling agent.

110. The pharmaceutical composition of claim 109, wherein said gelling agent is selected from the group consisting of hydroxypropyl methylcellulose and hydroxethylcellulose.

111. The pharmaceutical composition of claim 109, wherein a concentration of said gelling agent ranges between about 0.1 weight percentage and about 7 weight percentages of the total weight of the composition.

112. The pharmaceutical composition of claim 111, wherein a concentration of said gelling agent ranges between about 1 weight percentage and about 3 weight percentages.

113. The pharmaceutical composition of claim 80, further comprising at least one additive.

114. The pharmaceutical composition of claim 113, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

115. The pharmaceutical composition of claim 113, wherein a concentration of said additive ranges between about 1 weight percentage and about 5 weight percentages of the total weight of the composition.

116. The pharmaceutical composition of claim 80, packaged in a packaging material and identified in print, in or on the package, for use in the treatment of said skin disease or disorder.

117. The pharmaceutical composition of claim 116, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

118. A method for treating a skin disease or disorder in a subject in need thereof, the method comprising applying onto at least one biological surface affected by said skin disease or disorder of said subject a therapeutically effective amount of the pharmaceutical composition of claim 80.

119. The method of claim 118, wherein said at least one biological surface is selected from the group consisting of the skin of the face, an ear, a scalp, a neck, a forcarm, a back, a leg, an arms and a hand.

120. The method of claim 119, wherein said applying is performed between 1 and 4 times a day daily, for a time period that ranges between 20 days and 120 days.

121. The method of claim 120, wherein said applying is performed twice a day.

122. The method of claim 121, wherein said time period ranges between 30 and 60 days.

123. The method of claim 118, wherein said subject is a human.

124. The method of claim 118, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

125. The method of claim 124, wherein said skin disease or disorder is actinic keratosis.

126. The method of claim 118, wherein said pharmaceutical composition further comprises at least one penetration modifier.

127. The method of claim 126, wherein said penetration modifier is selected from the group consisting of diethylene glycol monomethyl ether, urea, glycerine, polysorbate 80, a hyaluronic acid or a salt thereof having a molecular weight less than about 150,000 Daltons, a hyaluronic acid or a salt thereof having a molecular weight greater than about 750,000 Daltons, and any mixture thereof.

128. The method of claim 127, wherein said at least one penetration modifier comprises diethylene glycol monoethyl ether.

129. The method of claim 128, wherein a concentration of said diethylene glycol monoethyl ether ranges between about 5 weight percentages and about 15 weight percentages of the total weight of said composition.

130. The method of claim 129, wherein said concentration of said diethylene glycol monoethyl ether ranges between about 5 weight percentages and about 10 weight percentages.

131. The method of claim 127, wherein said at least one penetration modifier comprises urea.

132. The method of claim 131, wherein a concentration of said urea ranges between about 5 weight percentages and about 15 weight percentages of the total weight of said composition.

133. The method of claim 132, wherein said concentration of said urea ranges between about 5 weight percentages and about 10 weight percentages.

134. The method of claim 127, wherein said composition further comprises at least one pH-stabilizing agent.

135. The method of claim 134, wherein said at least one pH stabilizing agent is selected from the group consisting of a hydroxyacid, allantoin, hydrochloric acid, a buffer system, an antioxidant and any mixture thereof.

136. The method of claim 126, wherein said at least one penetration modifier comprises glycerine.

137. The method of claim 136, wherein a concentration of said glycerine ranges between about 20 weight percentages and about 50 weight percentages of the total weight of said composition.

138. The method of claim 136, wherein said at least one penetration modifier further comprises diethylene glycol monoethyl ether.

139. The method of claim 138, wherein a concentration of said diethylene glycol monoethyl ether is about 10 weight percentages of the total weight of said composition.

140. The method of claim 138, wherein a concentration of said glycerine ranges between about 20 weight percentages and about 40 weight percentages, and wherein a concentration of said diethylene glycol monoethyl ether ranges between about 5 weight percentages and about 15 weight percentages.

141. The method of claim 126, wherein said at least one penetration modifier comprises polysorbate 80.

142. The method of claim 141, wherein a concentration of said polysorbate 80 ranges between about 1 weight percentage and about 5 weight percentages of the total weight of said composition.

143. The method of claim 142, wherein said concentration of said polysorbate ranges between about 2 weight percentages and about 3 weight percentages.

144. The method of claim 126 wherein said at least one penetration modifier is hyaluronic acid selected from the group consisting of a hyaluronic acid or a salt thereof having a molecular weight less than about 150,000 and a hyaluronic acid or a salt thereof having a molecular weight greater than about 750,000.

145. The method of claim 144, wherein a concentration of said hyaluronic acid ranges between about 1 weight percentage and about 3 weight percentages of the total weight of said composition.

146. The method of claim 145, wherein said concentration of said hyaluronic acid is about 2.5 weight percentages.

147. The method of claim 118, wherein said pharmaceutically acceptable salt is a sodium salt.

148. The method of claim 118, wherein a concentration of said diclofenac or said pharmaceutically acceptable salt ranges between about 1 weight percentage and about 5 weight percentages of the total weight of said composition.

149. The method of claim 148, wherein a concentration of said diclofenac or said pharmaceutically acceptable salt thereof is about 3 weight percentages.

150. The method of claim 118, wherein said composition is formulated in a form selected from the group consisting of a gel, a cream, an ointment, a paste, a lotion, a milk, a suspension, an aerosol, a spray, a foam, a serum, a swab, a pledglet, a pad and a patch.

151. The method of claim 150, wherein said composition is formulated in the form of a gel.

152. The method of claim 151, wherein said composition further comprises a gelling agent.

153. The method of claim 152, wherein said gelling agent is selected from the group consisting of hydroxypropyl methylcellulose and hydroxethylcellulose.

154. The method of claim 152, wherein a concentration of said gelling agent rages between about 0.1 weight percentage and about 7 weight percentages of the total weight of the composition.

155. The method of claim 154, wherein a concentration of said gelling agent ranges between about 1 weight percentage and about 3 weight percentages.

156. The method of claim 118, wherein said composition further comprises at least one additive.

157. The method of claim 156, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

158. The method of claim 157, wherein a concentration of said additive ranges between about 1 weight percentage and about 5 weight percentages.

159. A pharmaceutical composition, identified for use in the treatment of a skin disease or disorder, comprising, as a sole active ingredient, a non-steroidal anti-inflammatory drug or a pharmaceutically acceptable salt thereof, and a pharmaceutically acceptable carrier, the composition being essentially free of hyaluronic acid or a salt thereof having a molecular weight of between 150,000 and 750,000 Daltons.

160. The pharmaceutical composition of claim 159, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

161. The pharmaceutical composition of claim 160, wherein said skin disease or disorder is actinic keratosis.

162. The pharmaceutical composition of claim 159, further comprising at least one penetration modifier.

163. The pharmaceutical composition of claim 162, wherein said penetration modifier is selected from the group consisting of diethylene glycol monomethyl ether, urea, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof.

164. The pharmaceutical composition of claim 163, wherein said at least one penetration modifier comprises diethylene glycol monoethyl ether.

165. The pharmaceutical composition of claim 164, wherein a concentration of said diethylene glycol monoethyl ether ranges between about 5 weight percentages and about 15 weight percentages of the total weight of the composition.

166. The pharmaceutical composition of claim 165, wherein said concentration of said diethylene glycol monoethyl ether ranges between about 5 weight percentages and about 10 weight percentages.

167. The pharmaceutical composition of claim 163, wherein said at least one penetration modifier comprises urea.

168. The pharmaceutical composition of claim 167, wherein a concentration of said urea ranges between about 5 weight percentages and about 15 weight percentages of the total weight of the composition.

169. The pharmaceutical composition of claim 168, wherein said concentration of said urea ranges between about 5 weight percentages and about 10 weight percentages.

170. The pharmaceutical composition of claim 167, further comprising at least one pH-stabilizing agent.

171. The pharmaceutical composition of claim 170, wherein said at least one pH-stabilizing agent is selected from the group consisting of a hydroxyacid, allantoin, hydrochloric acid, a buffer system, an antioxidant and any mixture thereof.

172. The pharmaceutical composition of claim 163, wherein said at least one penetration modifier comprises glycerine.

173. The pharmaceutical composition of claim 172, wherein a concentration of said glycerine ranges between about 20 weight percentages and about 50 weight percentages of the total weight of the composition.

174. The pharmaceutical composition of claim 172, wherein said at least one penetration modifier further comprises diethylene glycol monoethyl ether.

175. The pharmaceutical composition of claim 174, wherein a concentration of said diethylene glycol monoethyl ether is about 10 weight percentages of the total weight of the composition.

176. The pharmaceutical composition of claim 174, wherein a concentration of said glycerine ranges between about 20 weight percentages and about 40 weight percentages, and wherein a concentration of said diethylene glycol monoethyl ether ranges between about 5 weight percentages and about 15 weight percentages.

177. The pharmaceutical composition of claim 163, wherein said at least one penetration modifier comprises polysorbate 80.

178. The pharmaceutical composition of claim 177, wherein a concentration of said polysorbate 80 ranges between about 1 weight percentage and about 5 weight percentages of the total weight of the composition.

179. The pharmaceutical composition of claim 178, wherein said concentration of said polysorbate 80 ranges between about 2 weight percentages and about 3 weight percentages.

180. The pharmaceutical composition of claim 163, wherein said at least one penetration modifier is a hyaluronic acid selected from the group consisting of hyaluronic acid or a salt thereof having a molecular weight of less than about 150,000 and hyaluronic acid or a salt thereof having a molecular weight greater than about 750,000.

181. She pharmaceutical composition of claim 180, wherein a concentration of said hyaluronic acid ranges between about 1 weight percentage and about 3 weight percentages of the total weight of the composition.

182. The pharmaceutical composition of claim 181, wherein said concentration of said hyaluronic acid is about 2.5 weight percentages.

183. The pharmaceutical composition of claim 159, wherein said non-steroidal anti-inflammatory drug is selected from the group consisting of diclofenac, oxicams, piroxicam, meloxicam, isoxicam, tenoxicam, sudoxicam, CP-14,304, salicylates, aspirin, disalcid, benorylate, trilisate, safapryn, solprin, diflunisal, fendosal, acetic acid derivatives, fenclofenac, indomethacin, sulindac, tolmetin, isoxepac, furofenac, tiopinac, zidometacin, acematacin, fentiazac, zomepirac, clindanac, oxcpinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, Suprofen, alminoprofen, tiaprofen, pyrazoles, phenylbutazone, oxyphenbutazonc, feprazone, azapropazone, trimethazone and derivatives, esters, salts and mixtures thereof, and combinations thereof.

184. The pharmaceutical composition of claim 183, wherein said non-steroidal anti-inflammatory drug is diclofenac or a pharmaceutically acceptable salt thereof.

185. The pharmaceutical composition of claim 184, wherein said pharmaceutically acceptable salt is a sodium salt.

186. The pharmaceutical composition of claim 159, wherein a concentration of said non-steroidal anti-inflamatory drug ranges between about 1 weight percentage and about 5 weight percentages of the total weight of the composition.

187. The pharmaceutical composition of claim 159, being formulated in a form selected from the group consisting of a gel, a cream, an ointment, a paste, a lotion, a milk, a suspension, an aerosol, a spray, a foam, a scrum, a swab, a pledglet, a pad and a patch.

188. The pharmaceutical composition of claim 187, being formulated in the form of a gel.

189. The pharmaceutical composition of claim 188, further comprising a gelling agent.

190. The pharmaceutical composition of claim 189, wherein said gelling agent is selected from the group consisting of hydroxypropyl methylcellulose and hydroxethylcellulose.

191. The pharmaceutical composition of claim 189, wherein a concentration of said gelling agent ranges between about 0.1 weight percentage and about 7 weight percentages of the composition.

192. The pharmaceutical composition of claim 191, wherein a concentration of said gelling agent ranges between about 1 weight percentage and about 3 weight percentages.

193. The pharmaceutical composition of claim 159, further comprising at least one additive.

194. The pharmaceutical composition of claim 193, wherein said additive is selected from the group consisting of a moisturizing agent, an emollient, a humectant, a deodorant agent, an antiperspirant, a pH adjusting agent, a preservative, an emulsifier, an occlusive agents a solubilizing agent, a colorant, and a surfactant.

195. The pharmaceutical composition of claim 193, wherein a concentration of said additive ranges between about 1 weight percentage and about 5 weight percentages.

196. The pharmaceutical composition of claim 159, packaged in a packaging material and identified in print, in or on the package, for use in the treatment of said skin disease or disorder.

197. The pharmaceutical composition of claim 196, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

198. A method for treating a skin disease or disorder in a subject in need thereof, the method comprising applying onto at least one biological surface affected by said skin disease or disorder of said subject a therapeutically effective amount of the pharmaceutical composition of claim 159.

199. The method of claim 198, wherein said at least one biological surface is selected from the group consisting of the skin of the face, an ear, a scalp, a neck, a forcarm, a back, a leg, an arms and a hand.

200. The method of claim 198, wherein said applying is performed between 1 and 4 times a day, for a time period that ranges between 20 days and 120 days.

201. The method of claim 200, wherein said applying is performed twice a day.

202. The method of claim 201, wherein said time period ranges between 30 and 60 days.

203. The method of claim 198, wherein said subject is a human.

204. The method of claim 198, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

205. The method of claim 204, wherein said skin disease or disorder is actinic keratosis.

206. The method of claim 198, wherein said composition further comprises at least one penetration modifier.

207. The method of claim 206, wherein said penetration modifier is selected from the group consisting of diethylene glycol monomethyl ether, urea, glycerine, polysorbate 80, a hyaluronic acid or a salt thereof having a molecular weight of less than about 150,000 Daltons, a hyaluronic acid or a salt thereof having a molecular weight greater than about 750,000 Daltons, and any mixture thereof.

208. The method of claim 207, wherein said at least one penetration modifier comprises diethylene glycol monoethyl ether.

209. The method of claim 208, wherein a concentration of said diethylene glycol monoethyl ether ranges between about 5 weight percentages and about 15 weight percentages of the total weight of said composition.

210. The method of claim 209, wherein a concentration of said diethylene glycol monoethyl ether ranges between about 5 weight percentages and about 10 weight percentages.

211. The method of claim 207, wherein said at least one penetration modifier comprises urea.

212. The method of claim 211, wherein a concentration of said urea ranges between about 5 weight percentages and about 15 weight percentages of the total weight of said composition.

213. The method of claim 212, wherein a concentration of said urea ranges between about 5 weight percentages and about 10 weight percentages.

214. The method of claim 211 wherein said composition further comprises at least one pH-stabilizing agent.

215. The method of claim 214, wherein said at least one pH stabilizing agent is selected from the group consisting of a hydroxyacid, allantoin, hydrochloric acid, a buffer system, an antioxidant and any mixture thereof.

216. The method of claim 207, wherein said at least one penetration modifier comprises glycerine.

217. The method of claim 216, wherein a concentration of said glycerine ranges between about 20 weight percentages and about 50 weight percentages of the total weight of said composition.

218. The method of claim 216, wherein said at least one penetration modifier further comprises diethylene glycol monoethyl ether.

219. The method of claim 218, wherein said concentration of said diethylene glycol monoethyl ether is about 10 weight percentages.

220. The method of claim 218, wherein a concentration of said glycerine ranges between about 20 weight percentages and about 40 weight percentages, and wherein a concentration of said diethylene glycol monoethyl ether ranges between about 5 weight percentages and about 15 weight percentages.

221. The method of claim 207, wherein said at least one penetration modifier comprises polysorbate 80.

222. The method of claim 221, wherein a concentration of said polysorbate 80 ranges between about 1 weight percentage and about 5 weight percentages of the total weight of said composition.

223. The method of claim 222, wherein said concentration of said polysorbate ranges between about 2 weight percentages and about 3 weight percentages.

224. The method of claim 207 wherein said at least one pension modifier is a hyaluronic acid selected from the group consisting of a hyaluronic acid or a salt thereof having a molecular weight of less than about 150,000 and a hyaluronic acid or a salt thereof having a molecular weight greater than about 750,000.

225. The method of claim 224, wherein a concentration of said hyaluronic acid ranges between about 1 weight percentage and about 3 weight percentages of the total weight of said composition.

226. The method of claim 225, wherein said concentration of said hyaluronic acid is about 2.5 weight percentages.

227. The method of claim 198, wherein said non-steroidal anti-inflammatory drug is selected from the group consisting of diclofenac, oxicams, piroxicam, meloxicam, isoxicam, tenoxicam, sudoxicam, CP-14,304, salicylates, aspirin, disalcid, benorylate, trilisate, safapryn, solprin, diflunisal, fendosal, acetic acid derivatives, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof, and combinations thereof.

228. The method of claim 227, wherein said non-steroidal anti-inflammatory drug is diclofenac or a pharmaceutically acceptable salt thereof.

229. The method of claim 228 wherein said pharmaceutically acceptable salt is a sodium salt.

230. The method of claim 198, wherein a concentration of said non-steroidal anti-inflammatory drug ranges between about 1 weight percentage and about 5 weight percentages of the total weight of said composition.

231. The method of claim 230, wherein said concentration of said non-steroidal anti-inflammatory drug is about 3 weight percentages.

232. The method of claim 207, wherein said composition is formulated in a form selected from the group consisting of a gel, a cream, an ointment, a paste, a lotion, a milk, a suspension, an aerosol, a spray, a foam, a serum, a swab, a pledglet, a pad and a patch.

233. The method of claim 198, wherein said composition is formulated in the form of a gel.

234. The method of claim 233, wherein said composition further comprises a gelling agent.

235. The method of claim 234, wherein said gelling agent is selected from the group consisting of hydroxypropyl methylcellulose and hydroxethylcellulose.

236. The method of claim 234, wherein a concentration of said gelling agent ranges between about 0.1 weight percentage and about 7 weight percentages of the total weight of said composition.

237. The method of claim 236, wherein a concentration of said gelling agent ranges between about 1 weight percentage and about 5 weight percentages.

238. The method of claim 198, wherein said composition further comprises at least one additive.

239. The method of claim 238, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

240. The method of claim 239, wherein a concentration of said additive ranges between about 1 weight percentage and about 5 weight percentages.

241. A pharmaceutical composition, identified for use in the treatment of a skin disease or disorder, comprising, as an active ingredient, a non-steroidal anti-inflammatory drug or a pharmaceutically acceptable salt thereof, and a pharmaceutically acceptable carrier, the composition being essentially free of hyaluronic acid or a salt thereof having a molecular weight of between 150,000 and 750,000 Daltons and is further being devoid of alpha-difluoromethylornithine.

242. The pharmaceutical composition of claim 241, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

243. The pharmaceutical composition of claim 242, wherein said skin disease or disorder is actinic keratosis.

244. The pharmaceutical composition of claim 241, further comprising at least one penetration modifier.

245. The pharmaceutical composition of claim 244, wherein said penetration modifier is selected from the group consisting of diethylene glycol monomethyl ether, urea, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof.

246. The pharmaceutical composition of claim 245, wherein said at least one penetration modifier comprises diethylene glycol monoethyl ether.

247. The pharmaceutical composition of claim 246, wherein a concentration of said diethylene glycol monoethyl ether ranges between about 5 weight percentages and about 15 weight percentages of the total weight of the composition.

248. The pharmaceutical composition of claim 247, wherein said concentration of said diethylene glycol monoethyl ether ranges between about 5 weight percentages and about 10 weight percentages.

249. The pharmaceutical composition of claim 245, wherein said at least one penetration modifier comprises urea.

250. The pharmaceutical composition of claim 249, wherein a concentration of said urea ranges between about 5 weight percentages and about 15 weight percentages of the total weight of the composition.

251. The pharmaceutical composition of claim 250, wherein said concentration of said urea ranges between about 5 weight percentages and about 10 weight percentages.

252. The pharmaceutical composition of claim 249, further comprising at least one pH-stabilizing agent.

253. The pharmaceutical composition of claim 252, wherein said at least one pH stabilizing agent is selected from the group consisting of a hydroxyacid, allantoin, hydrochloric acid, a buffer system, an antioxidant and any mixture thereof.

254. The pharmaceutical composition of claim 245, wherein said at least one penetration modifier comprises glycerine.

255. The pharmaceutical composition of claim 254, wherein a concentration of said glycerine ranges between about 20 weight percentages and about 50 weight percentages of the total weight of the composition.

256. The pharmaceutical composition of claim 254, wherein said at least one penetration modifier further comprises diethylene glycol monoethyl ether.

257. The pharmaceutical composition of claim 256, wherein a concentration of said diethylene glycol monoethyl ether is about 10 weight percentages of the total weight of the composition.

258. The pharmaceutical composition of claim 256, wherein a concentration of said glycerine ranges between about 20 weight percentages and about 40 weight percentages, and wherein a concentration of said diethylene glycol monoethyl ether ranges between about 5 weight percentages and about 15 weight percentages.

259. The pharmaceutical composition of claim 245, wherein said at least one penetration modifier comprises polysorbate 80.

260. The pharmaceutical composition of claim 259, wherein a concentration of said polysorbate 80 ranges between about 1 weight percentage and about 5 weight percentages of the total weight of the composition.

261. The pharmaceutical composition of claim 260, wherein said concentration of said polysorbate 80 ranges between about 2 weight percentages and about 3 weight percentages.

262. The pharmaceutical composition of claim 245, wherein said at least one penetration modifier is a hyaluronic acid selected from the group consisting of hyaluronic acid or a salt thereof having a molecular weight of less than about 150,000 and hyaluronic acid or a salt thereof having a molecular weight greater than about 750,000.

263. The pharmaceutical composition of claim 262, wherein a concentration of said hyaluronic acid ranges between about 1 weight percentage and about 3 weight percentages of the total weight of the composition.

264. The pharmaceutical composition of claim 263, wherein said concentration of said hyaluronic acid is about 2.5 weight percentages.

265. The pharmaceutical composition of claim 241, wherein said non-steroidal anti-inflammatory drug is selected from the group consisting of diclofenac, oxicams, piroxicam, meloxicam, isoxicam, tenoxicam, sudoxicam, CP-14,304, salicylates, aspirin, disalcid, benorylate, trilisate, safapryn, solprin, diflunisal, fendosal, acetic acid derivatives, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof, and combinations thereof.

266. The pharmaceutical composition of claim 265, wherein said non-steroidal anti-inflammatory drug is diclofenac or a pharmaceutically acceptable salt thereof.

267. The pharmaceutical composition of claim 266, wherein said non-steroidal anti-inflammatory drug is diclofenac sodium.

268. The pharmaceutical composition of claim 241, wherein a concentration of said non-steroidal anti-inflammatory drug ranges between about 1 weight percentage and about 5 weight percentages of the total weight of the composition.

269. The pharmaceutical composition of claim 241, being formulated in a form selected from the group consisting of a gel, a cream, an ointment, a paste, a lotion, a milk, a suspension, an aerosol, a spray, a foam, a serum, a swab, a pledglet, a pad and a patch.

270. The pharmaceutical composition of claim 269, being formulated in the form of a gel.

271. The pharmaceutical composition of claim 270, further comprising a gelling agent.

272. She pharmaceutical composition of claim 271, wherein said gelling agent is selected from the group consisting of hydroxypropyl methylcellulose and hydroxethylcellulose.

273. The pharmaceutical composition of claim 271, wherein a concentration of said gelling agent ranges between about 0.1 weight percentage and about 7 weight percentages of the composition.

274. The pharmaceutical composition of claim 273, wherein a concentration of said gelling agent ranges between about 1 weight percentage and about 3 weight percentages.

275. The pharmaceutical composition of claim 241, further comprising at least one additive.

276. The pharmaceutical composition of claim 275, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

277. The pharmaceutical composition of claim 275, wherein a concentration of said additive ranges between about 1 weight percentage and about 5 weight percentages.

278. The pharmaceutical composition of claim 241, packaged in a packaging material and identified in print, in or on the package, for use in the treatment of said skin disease or disorder.

279. The pharmaceutical composition of claim 278, wherein said skin disease or disorder is selected from the group consisting of basal cell carcinoma squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fez lesions, and hair loss in pregnancy.

280. A method for treating a skin disease or disorder in a subject in need thereof, the method comprising applying onto at least one biological surface affected by said skin disease or disorder of said subject a therapeutically effective amount of the pharmaceutical composition of claim 241.

281. The method of claim 280, wherein said at least one biological surface is selected from the group consisting of the skin of the face, an ear, a scalp, a neck, a forearm, a back, a leg, an arms and a hand.

282. The method of claim 280, wherein said applying is performed between 1 and 4 times a day, for a time period that ranges between 20 days and 120 days.

283. The method of claim 282, wherein said applying is performed twice a day.

284. The method of claim 283, wherein said time period ranges between 30 and 60 days.

285. The method of claim 280, wherein said subject is a human.

286. The method of claim 280, wherein said skin disease or disorder is selected from the group consisting of basal cell carcinoma, squamous cell tumor, cutaneous metastatic breast cancer, primary and metastatic melanoma in the skin, malignancies and tumors in the skin, genital warts, psoriasis, corns on the feet, actinic keratosis, liver spots, skin lesions, fungal lesions, and hair loss in pregnancy.

287. The method of claim 286, wherein said skin disease or disorder is actinic keratosis.

288. The method of claim 280, wherein said composition further comprises at least one penetration modifier.

289. The method of claim 288, wherein said penetration modifier is selected from the group consisting of diethylene glycol monomethyl ether, urea, glycerine, polysorbate 80, a hyaluronic acid or a salt thereof having a molecular weight of less than about 150,000 Daltons, a hyaluronic acid or a salt thereof having a molecular weight greater than about 750,000 Daltons, and any mixture thereof.

290. The method of claim 289, wherein said at least one penetration modifier comprises diethylene glycol monoethyl ether.

291. The method of claim 290, wherein a concentration of said diethylene glycol monoethyl ether ranges between about 5 weight percentages and about 15 weight percentages of the total weight of said composition.

292. The method of claim 291, wherein a concentration of said diethylene glycol monoethyl ether ranges between about 5 weight percentages and about 10 weight percentages.

293. The method of claim 289, wherein said at least one penetration modifier comprises urea.

294. The method of claim 293, wherein a concentration of said urea ranges between about 5 weight percentages and about 15 weight percentages of the total weight of said composition.

295. The method of claim 294, wherein a concentration of said urea ranges between about 5 weight percentages and about 10 weight percentages.

296. The method of claim 293, wherein said composition further comprises at least one pH-stabilizing agent.

297. The method of claim 296, wherein said at least one pH stabilizing agent is selected from the group consisting of a hydroxyacid, allantoin, hydrochloric acid, a buffer system, an antioxidant and any mixture thereof.

298. The method of claim 289, wherein said at least one penetration modifier comprises glycerine.

299. The method of claim 298, wherein a concentration of said glycerine ranges between about 20 weight percentages and about 50 weight percentages of the total weight of said composition.

300. The method of claim 298, wherein said at least one penetration modifier further comprises diethylene glycol monoethyl ether.

301. The method of claim 300, wherein said concentration of said diethylene glycol monoethyl ether is about 10 weight percentages.

302. The method of claim 300, wherein a concentration of said glycerine ranges between about 20 weight percentages and about 40 weight percentages, and wherein a concentration of said diethylene glycol monoethyl ether ranges between about 5 weight percentages and about 15 weight percentages.

303. The method of claim 289, wherein said at least one penetration modifier comprises polysorbate 80.

304. The method of claim 303, wherein a concentration of said polysorbate 80 ranges between about 1 weight percentage and about 5 weight percentages of the total weight of said composition.

305. The method of claim 304, wherein said concentration of said polysorbate ranges between about 2 weight percentages and about 3 weight percentages.

306. The method of claim 289 wherein said at least one penetration modifier is a hyaluronic acid selected from the group consisting of a hyaluronic acid or a salt thereof having a molecular weight of less than about 150,000 and a hyaluronic acid or a salt thereof having a molecular weight greater than about 750,000.

307. The method of claim 306, wherein a concentration of said hyaluronic acid ranges between about 1 weight percentage and about 3 weight percentages of the total weight of said composition.

308. The method of claim 307, wherein said concentration of said hyaluronic acid is about 2.5 weight percentages.

309. The method of claim 280, wherein said non-steroidal anti-inflammatory drug is selected from the group consisting of diclofenac, oxicams, piroxicam, meloxicam, isoxicam, tenoxicam, sudoxicam, CP-14,304, salicylates, aspirin, disalcid, benorylate, trilisate, safapryn, solprin, diflunisal, fendosal, acetic acid derivatives, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof, and combinations thereof.

310. The method of claim 309, wherein said non-steroidal anti-inflammatory drug is diclofenac or a pharmaceutically acceptable salt thereof.

311. The method of claim 310 wherein said pharmaceutically acceptable salt is a sodium salt.

312. The method of claim 280, wherein a concentration of said non-steroidal anti-inflammatory drug ranges between about 1 weight percentage and about 5 weight percentages of the total weight of said composition.

313. The method of claim 289, wherein said composition is formulated in a form selected from the group consisting of a gel, a cream, an ointment, a paste, a lotion, a milk, a suspension, an aerosol, a spray, a foam, a serum, a swab, a pledglet, a pad and a patch.

314. The method of claim 280, wherein said composition is formulated in the form of a gel.

315. The method of claim 314, wherein said composition further comprises a gelling agent.

316. The method of claim 315, wherein said gelling agent is selected from the group consisting of hydroxypropyl methylcellulose and hydroxethylcellulose.

317. The method of claim 315, wherein a concentration of said gelling agent ranges between about 01 weight percentage and about 7 weight percentages of the total weight of said composition.

318. The method of claim 317, wherein said concentration of said gelling agent ranges between about 1 weight percentage and about 3 weight percentages.

319. The method of claim 280, wherein said composition further comprises at least one additive.

320. The method of claim 319, wherein said additive is selected from the group consisting of a moisturizing agent, an emollient, a humectant, a deodorant agent, an antiperspirant, a pH adjusting agent, a preservative, an emulsifier, an occlusive agent, a solubilizing agent, a colorant, and a surfactant.

321. The method of claim 320, wherein a concentration of said additive ranges between about 1 weight percentage and about 5 weight percentages.

322. A pharmaceutical composition identified for use in the treatment of a skin disease or disorder, consisting essentially of:
diclofenac sodium;
a gelling agent selected from the group consisting of hydroxypropyl methylcellulose and hydroxyethylcellulose;
at least one penetration modifier selected from the group consisting of diethylene glycol monomethyl ether, urea, glycerine, polysorbate 80, hyaluronic acid or a salt thereof having a molecular weight of less than about 150,000 Daltons, hyaluronic acid or a salt thereof having a molecular weight greater than about 750,000 Daltons, and any mixture thereof;
at least one additive; and
a pharmaceutically acceptable carrier.

323. The pharmaceutical composition of claim 322, wherein said at least one additive comprises a preservative.

324. The pharmaceutical composition of claim 323, wherein said preservative is benzyl alcohol.

325. The pharmaceutical composition of claim 324, wherein a concentration of said benzyl alcohol is about 1 weight percentage of the total weight of the composition.

326. The pharmaceutical composition of claim 322, wherein a concentration of said gelling agent ranges between about 1 weight percentage and about 3 weight percentages of the total weight of the composition.

327. The pharmaceutical composition of claim 322, wherein a concentration of said diclofenac sodium is about 3 weight percentages of the total weight of the composition.

328. The pharmaceutical composition of claim 322, wherein said at least one penetration modifier comprises urea, and said at least one additive comprises a pH-stabilizing agent selected from the group consisting of allantoin and a buffer solution.

329. The pharmaceutical composition of claim 328, wherein said pharmaceutically acceptable carrier comprises a polyalkylene glycol and water.
